(19) United States
(12) Patent Application Publication
(10) Pub. No.: US 2015/0283179 A1
(43) Pub. Date: Oct. 8, 2015

(54) SELECTING FETAL BOVINE SERUM FOR USE IN STEM CELL CULTURE MEDIA AND PREPARING STEM CELL CULTURE

(71) Applicant: PHARMICELL CO., LTD., Seoul (KR)

(72) Inventors: Yoon Mi Kang, Seongnam-si (KR); Ha Cheol Shin, Seongnam-si (KR); Yong Man Kim, Seongnam-si (KR); Wan Seok Joo, Seoul (KR); Hyun Soo Kim, Seoul (KR)

(73) Assignee: PHARMICELL CO., LTD., Seoul (KR)

(21) Appl. No.: 14/247,185

(22) Filed: Apr. 7, 2014

(51) Int. Cl. A61K 35/28

(52) U.S. Cl. CPC A61K 35/28

(57) ABSTRACT

A method of selecting a collection of cells for choosing a fetal bovine serum (FBS) to use in stem cell transplantation is provided. In addition, a method of selecting a fetal bovine serum (FBS) for use in culturing stem cells for transplantation is provided. Furthermore, a culture medium for culturing stem cells for transplantation and a method of stem cell transplantation are provided.

### BACKGROUND

The disclosure relates to culturing stem cells. More particularly, the disclosure relates to optimizing a culture medium for culturing stem cells and selecting stem cells for use in optimizing a culture medium.

Stem cells are known for their capabilities of differentiation to multiple types of tissues, provision of microenvironment suitable for regeneration of damaged tissues, and secretion of immunosuppressants or immune-regulatory molecules. Stem cells can be cultured in vitro using appropriate cell culture media which may comprise Fetal Bovine Serum (FBS).

### SUMMARY

In one aspect, the present invention provides a method of selecting a collection of cells for choosing a fetal bovine serum (FBS) to use in stem cell transplantation. The method may comprise providing a plurality of collections of cells, cells of each collection being of a different source from cells of another of the plurality of collections, monitoring growth of cells in the plurality of collections, providing information associated with each collection, and selecting a first one among the plurality of collections based on either or both of the monitoring of cell growth and further based on the information associated with each collection.

The information may be selected from the group consisting of the age of a subject from which the collection of cells was obtained, the weight of a subject from which the collection of cells was obtained, the gender of a subject from which the collection of cells was obtained, the type of tissues of the subject from which the collection of cells was obtained, information about the current and previous health condition of the subject from which the collection of cells was obtained, family medical history of the subject from which the collection of cells was obtained, and the number of culturing passages of the collection after the collection of cells was originally obtained from the subject.

In some embodiments, the selection of the first collection is based on that the first collection of cells is one of a least-growing group of collections, wherein the collections of the least-growing group are bottom 10% in cell growth among the plurality of collections. In some other embodiments, the selection of the first collection is based on that the first collection of cells is the least-growing collection in cell growth compared to the other collections. In still some other embodiments, the selection of the first collection is based on that the first collection of cells was obtained from a subject at the age of 80 or older. In still some other embodiments, the selection of the first collection is based on that the first collection of cells was obtained from bone marrow of the subject.

In still some other embodiments, the selection of the first collection is based on that the first collection of cells was obtained from a subject when the subject was medically diagnosed of a disease. In still some other embodiments, the selection of the first collection is based on that the first collection of cells was obtained from a subject having history of a disease selected from the group consisting of myocardial infarction, ischemic stroke, spinal cord injury, or liver cirrhosis. In certain embodiments, the selection of the first collection is based on that the first collection of cells is one of a least-growing group of collections, wherein the collections of the least-growing group are bottom 10% in cell growth among the plurality of collections, and obtained from a subject at the age of 80 or older.

In certain other embodiments, the selection of the first collection is based on that the first collection of cells is one of a least-growing group of collections, wherein the collections of the least-growing group are bottom 10% in cell growth among the plurality of collections and obtained from a subject when the subject was medically diagnosed of a disease. In still certain some other embodiments, the selection of the first collection is based on that the first collection of cells is one of a least-growing group of collections, wherein the collections of the least-growing group are bottom 10% in cell growth among the plurality of collections, and obtained from a subject having history of a disease selected from the group consisting of myocardial infarction, ischemic stroke, spinal cord injury, or liver cirrhosis. In still certain some other embodiments, the selection of the first collection is based on that the first collection of cells is one of a least-growing group of collections, wherein the collections of the least-growing group are bottom 10% in cell growth among the plurality of collections, obtained from a subject at the age of 80 or older; and obtained from a subject when the subject was medically diagnosed of a disease.

In another aspect, the present invention provides a method of selecting a fetal bovine serum (FBS) for use in culturing stem cells for transplantation. The method may comprise providing a collection of cells selected by one of the above-identified methods, providing a plurality of FBS samples, formulating a plurality of culture media, each comprising one of the plurality of FBS samples, culturing the cells of the selected collection in the plurality of culture media, monitoring growth of the cells of the selected collection in the plurality of culture media, based on monitoring, identifying one or more culture media, among the plurality of culture media, that has shown at least 1.5 fold cell growth after one culturing passage, and selecting at least one FBS that is included in the one or more identified culture media for use in culturing stem cells for transplantation.

In some embodiments, the method further comprises prior to selecting at least one FBS, identifying at least one culture medium, among the selected one or more culture media that has shown at least 142 fold cell growth after five culturing passages. In still some embodiments, the method further comprises prior to selecting at least one FBS, identifying at least one culture medium, among the selected one or more culture media that has shown at least 100 fold cell growth after five culturing passages. In still some embodiments, the method further comprises prior to selecting at least one FBS, identifying at least one culture medium, among the selected one or more culture media, in which the cells of the selected collection have shown a plastic-adherent, fibroblast-like morphology. In still some embodiments, the method further comprises prior to selecting at least one FBS, identifying at least one culture medium, among the selected one or more culture media, in which the cells of the selected collection have shown a high level of expression of CD105, and CD73 and a low level of expression of CD45, CD34, CD14. In still some embodiments, the method further comprises prior to selecting at least one FBS, identifying at least one culture medium, among the selected one or more culture media, in which the cells of the selected collection have potency to differentiate into at least one selected from the group consisting of osteoblast, adipocyte and cardiomyocyte. In some other embodiments, the identifying comprises further culturing the cells of the selected collection to determine if the cells differentiate into at least one of osteoblast, adipocyte and cardiomyocyte.

In still another aspect, the present invention provides a culture medium for culturing stem cells for transplantation. The culture medium may comprise an FBS selected by the above-identified method and a basal medium.

In still another aspect, the present invention provides a method of stem cell transplantation. The method may comprise providing mononuclear cells for culturing, providing a culture media comprising an FBS selected by the above-identified method, culturing the mononuclear cells in the culture media to produce mesenchymal stem cells, transplanting the produced mesenchymal stem cells to a patient. In some embodiments, the providing the mononuclear cells comprises obtaining the mononuclear cells from the same patient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates average growth rates of the tested pool of collections/groups of cells.

FIG. 2 illustrates the growth curve of T34 cells.

FIG. 3 illustrates the growth curve of T35 cells.

FIG. 4 illustrates cellular morphology of T34 cells is illustrated.

FIG. 5 illustrates cellular morphology of T35 cells is illustrated.

FIG. 6 illustrates the growth curve of T34 cells.

FIG. 7 illustrates the growth curve of T43 cells

FIG. 8 illustrates cellular morphology of T34 cells.

FIG. 9 illustrates cellular morphology of T43 cells.

FIG. 10 illustrates that T34 and T43 cells were efficiently differentiated to osteoblast.

FIG. 11 illustrates that T34 and T43 cells were efficiently differentiated to adipocyte.

FIG. 12 illustrates that T34 and T43 cells were efficiently differentiated to cardiomyocyte.

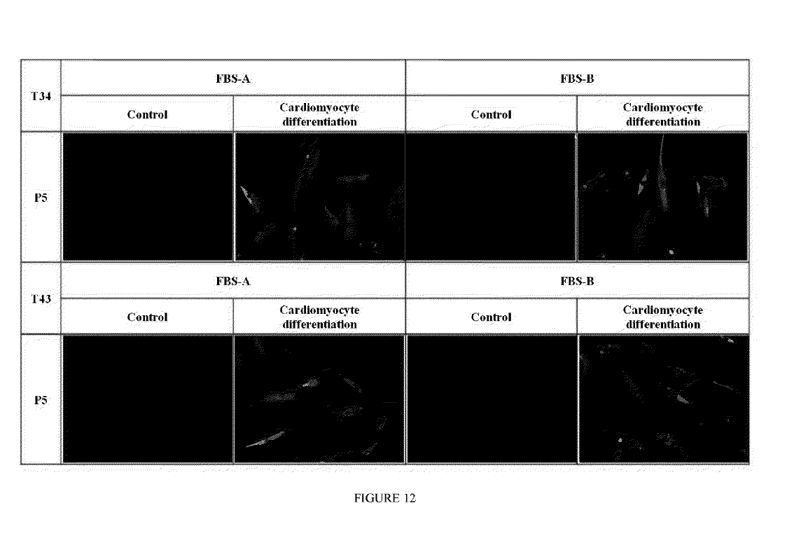

### DETAILED DESCRIPTION OF EMBODIMENTS

Various embodiments of the present invention will now be described in more detail with reference to the accompanying drawings. Embodiments described in the detailed description and drawings are not meant to be limiting. Other embodiments may be utilized, and changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

### Cell Therapy

A cell therapy is a process of introducing certain therapeutic cells into a tissue in order to treat a disease. Cell therapies target many clinical indications in multiple organs and by several modes of cell delivery. A cell therapy utilizes:
Stem cell or progenitor cell engraftment, differentiation, and long term replacement of damaged tissue. In this paradigm multipotent or unipotent stem cells differentiate into a specific cell type in a lab or after reaching the site of injury (via local or systemic administration). These cells then integrate into the site of injury, replacing damaged tissue, and thus facilitate improved function of the organ or tissue. An example of this is the use of cells to replace cardiomyocytes after myocardial infarction; and
Cells that have the capacity to release soluble factors such as cytokines, chemokines, and growth factors. These factors facilitate self-healing of the organ or region. This includes cells that naturally secrete the relevant therapeutic factors, or which undergo epigenetic changes or genetic engineering that causes the cells to release large quantities of a specific molecule. Examples of this include cells that secrete factors which facilitate angiogenesis, anti-inflammation, and anti-apoptosis.
   Autologous Cell Therapy Vs. Allogeneic Cell Therapy

There are two general classes of cell therapy approaches to treat patients. First, cells may be harvested from a patient and treated or expanded and introduced back into the same patient. This patient-specific, or autologous, method has historically been favored due to the lack of required immunologic matching. The second approach involves harvesting of cells from one, or a few donors that may be followed by expansion and banking of one or more doses, i.e. an allogeneic approach. This allogeneic approach utilizes cell types that would not elicit immune responses upon implantation in a patient. The cells isolated from an autologous or allogeneic source can be expanded in a manufacturing facility and/or cryopreserved for later manipulation or as therapeutic doses. Cell therapies generally require isolation of stem cells (either autologous or allogeneic), culturing the isolated stem cells, and transplantation of the cultured stem cells to the patient.

### Stem Cells

Adult stem cells are multi-potent cells that are committed to specific lineages. They can replenish dying cells and damaged tissues by multiplying through cell division and differentiating into a subset of cell types specific to its lineage. As such, they hold vast regenerative and therapeutic potential. Furthermore, their use in cell therapy may be less controversial as they can be harvested from various sources in humans whereas the use of human embryonic stem cells (hESCs) often entails destruction of human embryos. There have been significant advancements in clinical applications of neural, mesenchymal and hematopoietic stem cells.

### Mesenchymal Stem Cells (MSC)

Mesenchymal stem cells (SCs) are immunomodulatory, multipotent and fast proliferating. Increasingly, mesenchymal stem cells are being proposed as agents for cell-based therapies, due to their plasticity, established isolation procedures, and capacity for ex vivo expansion. Therapeutic targets of cell therapy using MSC (MSC therapy) may include myocardial infarction, central nervous system disorders, stroke, graft-versus-host disease (GVHD), Crohn's disease, spinal cord injury, multiple sclerosis, diabetes, osteoarthritis, osteogenesis imperfect and other diseases. In such a treatment, the mesenchymal stem cells can be obtained from a patient (i.e. autologous) or from a different individual who shares some genetic traits, thereby suitable for being a source of stem cells (i.e. allogeneic). There have been multiple clinical trials which prove safety and efficacy of cell therapy using mesenchymal stem cells.

### Importance of Stem Cell Culture System

Common challenges to the autologous and allogeneic approaches primarily reside in the preparation of stem cells for delivery (or transplantation to the patient). The number of cells that are firstly isolated from an autologous or allogeneic source may or may not be sufficient to be transplanted into a patient. Accordingly, culturing the isolated stem cells outside of a donor may be necessary, and occasionally one or more passaged-culturing would be needed in order to obtain a number of stem cells that is sufficient for one or more transplantation. In addition, the stem cells may need to maintain their stemness (i.e. the capability to be differentiated into multiple cell types) during the culturing passages. Therefore, once transplanted into a patient, the stem cells can be differentiated into various cell types depending on the patient's need.

Accordingly, in order to successfully operate cell therapy, establishment of a cell culture system that can produce a certain number of cells to satisfy a dosing requirement is necessary. Further, the cells need to be efficiently expanded in culture while retaining their proper cell characterization profile and efficacy.

One aspect of the present invention relates to a method of establishing an optimized cell culture system that allows stable growth and proliferation of primary stem cells (i.e. the stem cells isolated from an autologous or allogeneic source) while largely maintaining their innate biological characteristics (e.g. multipotency, secretion of soluble factors, and others).

### Factors for Stem Cell Culture

Cells isolated from various sources may vary from each other. For example, cellular characteristics such as health, growth rate, division date, and multipotency may differ depending on the donor's inherent and/or acquired conditions. More particularly, the biological characteristics of the isolated primary stem cells can be different based on age, gender, health condition, genetics, previous and current disease history, family history, and type and condition of tissue from which the stem cells are isolated. In addition, the culturing time and passage number may also be factors responsible for the change of biological characteristics of stem cells, especially related to any change in stemness of the cultured cells.

In addition to the foregoing, various environmental factors such as medium's composition, cell culture device's physical and chemical configuration, cell density, oxygen concentration, carbon dioxide concentration (and any other gases' concentrations if relevant to the cell growth), pH, temperature, osmolality, buffer, and many others can also lead to the change of cell's biological characteristics including loss or reduction of stemness of the cultured cells.

### Issues in Stem Cell Culture

Cell therapies utilize cells that are isolated from a donor. In some occasions, the cells that are isolated from the donor may not be sufficient in number to transplant to a patient. In such a case, the isolated primary cells are cultured in vitro in order to obtain a sufficient number of cells for the therapy. In addition, when the first trial of cell therapy is not successful and therefore an additional trial is in need, additional cells must be available for the repeat. If there is no suitable and optimized culture system, and therefore unable to provide enough cells, the cell therapy cannot be repeated. If there is no optimized cell culture system, it will lead to loss of all or substantial loss of primary cells. Consequently, an additional isolation of stem cells will become necessary. However, such would be difficult or would not be possible in some cases. The donor may not allow isolating stem cells multiple times. Especially, if the donor is not healthy, which is true in many cases of autologous cell therapy, it is difficult or not proper to conduct multiple procedures to isolate stem cells. Because the source of primary cells is generally limited, and because multiple isolations of stem cells may not be practical in many occasions, it is important to establish an optimized cell culture system for a successful cell therapy.

### Fetal Bovine Serum (FBS)

One important component of a stem cell culture medium is Fetal Bovine Serum (FBS). Typically, a stem cell culture media contains FBS in an amount of 10 to 20% by weigh of the overall culture media. FBS plays important roles such as providing necessary nutrients, growth factors, and adherence factors for growing stem cells. FBS includes many different factors, some of which allow or induce cell growth (growth factors) or help cell adherence and spreading (adhesion factors). Further, other factors such as vitamins, minerals, lipids, hormones, nutrients such as amino acids, nucleic acids, buffering agents such as albumin, inhibitors, and many others which influence cell growth are provided in FBS.

Many ingredients present in any given cell culture medium are synthesized, and therefore the quality of such synthetic ingredients is largely controllable and consistent. However, FBS is only obtained from an individual bovine fetus. Accordingly, the activity or efficacy of FBS varies depending on the source (i.e. individual bovine fetus). In general, companies producing FBS assign the same lot number to FBS produced from the same source. Accordingly, FBS having different lot numbers may have different compositions and activities. In order to optimize the cell culture medium and maintain the optimized condition, it is important to use FBS with good activity. However, as noted above, the FBS quality cannot be controlled. Further, no good method has been proposed to evaluate the efficacy of newly obtained FBS prior to use. Accordingly, every time the FBS lot number changes, meaning a new batch of FBS is used, researches generally conduct a test experiment to evaluate the efficacy of the new batch of FBS.

### Evaluation of New Batch of FBS

Evaluation of the efficacy of a new batch of FBS can be done by monitoring cell growth. In general, test cells of the same progeny are grown in a medium containing a new batch of FBS and in another medium containing an old batch of FBS. The old batch of FBS refers to the one that is being currently used or was previously used and is most likely one that was proven to be suitable for culturing the test cell type. The old batch of FBS is used as a control. If the cells cultured in a new batch of FBS show a similar or higher growth rate compared to the cells cultured in the old FBS, the new batch of FBS may be determined as being an efficient and suitable batch of FBS for continuously culturing the tested type of cells. However, if the growth of the cells cultured in the new batch of FBS is substantially lower than that of the cells grown in the old batch of FBS, the new batch of FBS would not be as efficient and active as the old batch of FBS and therefore may not be considered suitable for culturing the tested type of cells.

### Different Sensitivity to FBS by Individual Cells

The sensitivity to FBS may vary depending on cell types. Certain cells are more tolerant (or insensitive) to different batches of FBS than others. Therefore, such more tolerant cells would likely grow relatively consistently even when FBS changes in their culture medium. Generally speaking, cells that are relatively insensitive to different batches of FBS are generally healthy. However, some cells are more sensitive to FBS changes. Therefore, the growth of such sensitive cells will be substantially affected, often in a negative manner, when FBS from different lots is used. Such sensitive cells are often weak cells, meaning that they grow relatively slow compared to non-sensitive cells and do not divide frequently. Therefore, it needs an extra caution to select a good batch of FBS that is suitable for culturing cells that are sensitive to the FBS efficacy.

Primary stem cells isolated from an autologous source or an allogeneic donor will have different levels of sensitivity toward FBS. Especially, when the primary cells are isolated from a patient in an autologous therapy, the patient may have a condition(s) or disease(s) that deteriorates the health of the patient. Therefore, the patient may not be as healthy as an average non-patient donor. In such a case, stem cells isolated from the patient may not be as healthy as the same type of stem cells that are isolated from a healthy non-patient donor. Stem cells isolated from a patient often do not grow well in vitro relative to stem cells isolated from a healthy individual. Further, stem cells isolated from a patient may show a relatively higher sensitivity to the efficacy of cell culture medium, especially to FBS. Overall stem cells isolated from patient donors grow poorly or may not grow at all when cultured in vitro. Even if primary stem cells are grown relatively well in one batch of FBS, their growth would not be guaranteed in another batch of FBS. A similar high degree of sensitivity toward FBS may also be found in stem cells isolated from a healthy non-patient donor.

### Difficulty of Evaluating FBS Efficacy for Individual Stem Cells

Cells isolated from different sources would likely have different degrees of sensitivity to FBS. Therefore, it would be ideal to test several batches of FBS for each group of cells when they are firstly isolated from a source, and select a suitable batch of FBS in order to stably grow the cells in vitro. However, this would not be practical in cell therapies using stem cells as the number of stem cells isolated from a donor is quite limited to conduct such testing of multiple batches of FBS. Moreover, it is often be difficult to isolate stem cells again from the same donor, particularly a patient donor, if the first trial of culturing in vitro is not successful. Therefore, it may not be possible or at least is difficult to test multiple FBS for selecting an FBS that would fit the particular stem cells available for a cell therapy.

### Selecting Efficient FBS for Stem Cell Therapy

In view of the unpredictable sensitivity of cells to different batches of FBS, culturing stem cells for stem cell therapy is somewhat unpredictable. Also, in view of the difficult or impracticable nature of testing multiple FBS batches for stem cell therapy, successfully culturing stem cells for stem cell therapy would be very difficult. Further, in view of the generally higher sensitivity of stem cells of a patient donor to FBS, culturing stem cells for autologous stem cell therapy would be even more difficult. All of these involve the lack of a mechanism for finding an FBS batch that would allow a good growth of stem cells for use in stem cell therapy.

One aspect of the present invention provides a method of selecting a batch of FBS that would allow a relatively good growth of stem cells isolated from various sources. In accordance with embodiments of the invention, one or more batches of FBS having a high level of efficacy or activity for various stem cells are chosen in advance of isolating stem cells from a source. While the chosen FBS batches may not work for all stem cells in the same manner, the selection method provides significant advantages and also provides a mechanism for a stable supply of highly effective or active FBS batches for culturing stem cells for stem cell therapy. In accordance with embodiments of the invention, searching for proper FBS for particular stem cells after isolating from a source can be avoided.

### Test Cell Collections/Groups

Individual groups/collections of cells have varying sensitivity to FBS. Here, the term “individual group/collection of cells” refers to cells that are isolated from the same source (or donor) at the same time. The primary stem cells isolated from an autologous, patient source often show a higher degree of sensitivity to FBS as compared to cells isolated from an allogeneic, non-patient source. Therefore, such autologous primary stem cells may often be weaker or grow less than those from an allogeneic donor when they are grown in the same conditions. Accordingly, the selection of a batch of FBS for culturing such primary stem cells is important.

In one aspect, the present invention provides a method of selecting an FBS. The method may utilize one or more collections/groups of cells that are relatively sensitive to FBS as test cell collections/groups. In embodiments, test cell collections (or groups) refer to cell collections/groups that are used in test. The test cell groups are chosen from cells that have a relatively high sensitivity to FBS, and therefore would be representative of stem cells that are also sensitive to FBS such as autologous primary stem cells. One or more of test cell collections are cultured in one medium containing a new batch of FBS and in another medium containing an old FBS (that is proven efficient for stem cell culture), respectively, and the growth and health condition of the test cell collections in both media are monitored. If the new batch of FBS would allow the test cell collections to grow relatively well and healthy compared to the old, efficient FBS, the new batch of FBS will be considered suitable for culturing stem cells that are sensitive to FBS. The new batch of FBS can then be used for culturing other stem cells that may have high sensitivity to FBS, e.g. the stem cells that are isolated from a donor, especially from a patient.

When a mixture of mononuclear cells is first separated from a donor, for example, from bone marrow, by density fractionation or red blood cell lysis, the separated cells generally comprise largely hematopoietic cells and rare mesenchymal stem cells. Mesenchymal stem cells have the ability to adhere to plastic surfaces of tissue culture dishes or flasks. So, when the isolated mononuclear cells are cultured in vitro, mesenchymal stem cells adhere to the culture plastics and non-adherent hematopoietic cells are washed away. Therefore, after the initial culture of mononuclear cells, largely mesenchymal stem cells (MSC) remain and continuously grow. In one embodiment, mononuclear cells are used as test cells. In another embodiment, mesenchymal stem cells (MSC) which are isolated or grown out from the culturing of mononuclear cells are used as test cells.

The cells, e.g. mononuclear cells, may be isolated from various tissues such as bone marrow, adipose tissue, blood, cord blood, placenta, and more.

### Collection of Relevant Information

Test cell collections must have a relatively high degree of sensitivity toward FBS than other cells. Therefore, it is important to select cell collections having a high sensitivity to FBS in order to establish test cell collections. According to one embodiment, one or more test cell collections are selected among a plurality of candidate cell collections. For this purpose, first a lot of different cell collections are prepared. Each collection/group of cells is isolated from one donor and is used as a candidate cell collection/group. When preparing individual candidate cell collections, information related to the cells, which may be relevant to the condition of the cells, is obtained. For example, information obtained includes one or more of donor's age, weight, gender, health condition, previous and/or current medical history, family medical history, the particular tissue where the cells are isolated (i.e. source tissue) and the condition of source tissue. In addition, if the candidate cells were previously cultured in vitro, the period of culture and passage number may also be obtained. Once the information related to the condition of cells is collected, the information is compiled in a database.

### Growth Rates

In embodiments, once a plurality of candidate cell collections is prepared, these candidate cell collections are grown in any existing (or previously used) culture medium. The growth of individual candidate collections is monitored, and their growth rates are measured. One or more collections of cells showing relatively lower growth rates are selected. In embodiments, the collections showing lower than about 75%, about 70%, about 65%, about 60%, about 55%, about 50%, about 45%, about 40%, about 37%, about 35%, about 32%, about 30%, about 27%, about 25%, about 22%, about 20%, about 17%, about 15%, about 12%, about 10%, about 9%, about 8%, about 7%, about 6%, about 5%, about 4%, about 3%, about 2%, and about 1% growth rate may be selected as primary candidates.

### Ranking of Collected Cells

In addition to the growth rate, in embodiments, each group/collection of candidate cells may be ranked based on the information related to the donor and the originating source tissues. For example, the candidate cells may be ranked based on the donor's age or age range. In some other examples, the candidate cells may be ranked based on the previous medical history or current medical condition of the donor. Further, the candidate cells may be ranked based on the source tissue. For example, if a disease targeted in a cell therapy is related to a certain tissue, the candidate cells isolated from the same tissue of the target disease may be ranked higher than other candidate cells that were isolated from different tissues.

Also, in an additional example, the candidate cells may be ranked based on the donor's health condition. In embodiments, two or more pieces of information may be considered in ranking the candidate cells. For example, the candidate cells obtained from a relatively aged donor whose health is not normal would be ranked differently from (e.g., lower than) the candidate cells obtained from a donor who is younger and healthy. Further, for example, if there are candidate cells that have already been cultured in vitro for a certain period, these cells would be ranked differently from (e.g., lower than) the other cells which have not undergone in vitro culturing. This collected information may be considered along with the growth rate data when selecting test cell collections. More particularly, the candidate cells associated with the factors that are believed to possibly negatively affect the growth condition of the cells would be preferred over the cells that are not associated with such information. Therefore, in one example, the candidate cells isolated from a more aged donor would be preferred as a test cell collection/group over the candidate cells isolated from a younger donor. Further, if a candidate cell collection/group is from a donor having a certain pathological condition or disease, such a candidate cell collection/group can be preferred over the other candidate collections isolated from health donors.

In another example where more than one types of information is considered, a candidate cell collection/group isolated from an aged donor having a clinical condition or disease may be preferred over other candidate collections that are isolated from a younger donor having no pathologic condition or disease.

### Selecting Test Cell Collections

In embodiments, to select test cell collections from a plurality of candidate cell collections, the growth rate and the information associated with the candidate cells may be considered together. In one embodiment, the growth rate is considered together with one or more pieces of information related to the collected cells, for example, donor's age, weight, gender, health condition, previous and/or current medical history, family medical history. In one embodiment, source tissue and the condition of source tissue, and the period of previous culture and passage number are also considered. For instance, the growth rate may be considered along with the age and health condition of the donor for the selection of test cell collections from the candidate cell collections. Also, the growth rate may be considered together with the donor's age, health condition, and the source tissue to determine test cell collections. Some non-limiting and illustrative examples are provided below.

### Growth Rate and Age

In embodiments, one can select several candidate cell collections based on growth rate, and further narrow down the number by selecting only those satisfying one or more other criteria. In one embodiment, one can select the candidate cells showing about 10% or lower in growth rate, and further select the cells that are isolated from a donor of the age of 45 or higher. In another embodiment, one can select the candidate cells showing about 10% or lower in growth rate, and further select the cells that are isolated from a donor of the age of 55 or higher. In still another embodiment, one can select the candidate cells showing about 10% or lower in growth rate, and further select the cells that are isolated from a donor of the age of 65 or higher. In still another embodiment, one can select the candidate cells showing about 10% or lower in growth rate, and further select the cells that are isolated from a donor of the age of 75 or higher. In still another embodiment, one can select the candidate cells showing about 10% or lower in growth rate, and further select the cells that are isolated from a donor of the age of 80 or higher.

In still other embodiments, one can select the candidate cells showing about 75% or lower, about 70% or lower, about 65% or lower, about 60% or lower, about 55% or lower, about 50% or lower, about 45% or lower, about 40% or lower, about 37% or lower, about 35% or lower, about 32% or lower, about 30% or lower, about 27% or lower, about 25% or lower, about 22% or lower, about 20% or lower, about 17% or lower, about 15% or lower, about 12% or lower, about 10% or lower, about 9% or lower, about 8% or lower, about 7% or lower, about 6% or lower, about 5% or lower, about 4% or lower, about 3% or lower, about 2% or lower, or about 1% or lower in growth rate, and further narrow down to the cells that are isolated from a donor of the age of 20 or higher, 21 or higher, 22 or higher, 23 or higher, 24 or higher, 25 or higher, 26 or higher, 27 or higher, 28 or higher, 29 or higher, 30 or higher, 31 or higher, 32 or higher, 33 or higher, 34 or higher, 35 or higher, 36 or higher, 37 or higher, 38 or higher, 39 or higher, 40 or higher, 41 or higher, 42 or higher, 43 or higher, 44 or higher, 55 or higher, 46 or higher, 47 or higher, 48 or higher, 49 or higher, 50 or higher, 51 or higher, 52 or higher, 53 or higher, 54 or higher, 55 or higher, 56 or higher, 57 or higher, 58 or higher, 59 or higher, 60 or higher, 61 or higher, 62 or higher, 63 or higher, 64 or higher, 65 or higher, 66 or higher, 67 or higher, 68 or higher, 69 or higher, 70 or higher, 71 or higher, 72 or higher, 73 or higher, 74 or higher, 75 or higher, 76 or higher, 77 or higher, 78 or higher, 79 or higher, 80 or higher, 81 or higher, 82 or higher, 83 or higher, 84 or higher, 85 or higher, 86 or higher, 87 or higher, 88 or higher, 89 or higher, 90 or higher, 91 or higher, 92 or higher, 93 or higher, 94 or higher, or 95 or higher.

In still other embodiments, one can select the candidate cells showing about 75% or lower, about 70% or lower, about 65% or lower, about 60% or lower, about 55% or lower, about 50% or lower, about 45% or lower, about 40% or lower, about 37% or lower, about 35% or lower, about 32% or lower, about 30% or lower, about 27% or lower, about 25% or lower, about 22% or lower, about 20% or lower, about 17% or lower, about 15% or lower, about 12% or lower, about 10% or lower, about 9% or lower, about 8% or lower, about 7% or lower, about 6% or lower, about 5% or lower, about 4% or lower, about 3% or lower, about 2% or lower, or about 1% or lower in growth rate, and further narrow down to the cells that are isolated from a donor of the age of 20 or lower, 21 or lower, 22 or lower, 23 or lower, 24 or lower, 25 or lower, 26 or lower, 27 or lower, 28 or lower, 29 or lower, 30 or lower, 31 or lower, 32 or lower, 33 or lower, 34 or lower, 35 or lower, 36 or lower, 37 or lower, 38 or lower, 39 or lower, 40 or lower, 41 or lower, 42 or lower, 43 or lower, 44 or lower, 55 or lower, 46 or lower, 47 or lower, 48 or lower, 49 or lower, 50 or lower, 51 or lower, 52 or lower, 53 or lower, 54 or lower, 55 or lower, 56 or lower, 57 or lower, 58 or lower, 59 or lower, 60 or lower, 61 or lower, 62 or lower, 63 or lower, 64 or lower, 65 or lower, 66 or lower, 67 or lower, 68 or lower, 69 or lower, 70 or lower, 71 or lower, 72 or lower, 73 or lower, 74 or lower, 75 or lower, 76 or lower, 77 or lower, 78 or lower, 79 or lower, 80 or lower, 81 or lower, 82 or lower, 83 or lower, 84 or lower, 85 or lower, 86 or lower, 87 or lower, 88 or lower, 89 or lower, 90 or lower, 91 or lower, 92 or lower, 93 or lower, 94 or lower, or 95 or lower.

In still other embodiments, one can select the candidate cells showing about 75% or lower, about 70% or lower, about 65% or lower, about 60% or lower, about 55% or lower, about 50% or lower, about 45% or lower, about 40% or lower, about 37% or lower, about 35% or lower, about 32% or lower, about 30% or lower, about 27% or lower, about 25% or lower, about 22% or lower, about 20% or lower, about 17% or lower, about 15% or lower, about 12% or lower, about 10% or lower, about 9% or lower, about 8% or lower, about 7% or lower, about 6% or lower, about 5% or lower, about 4% or lower, about 3% or lower, about 2% or lower, or about 1% or lower in growth rate, and further narrow down to the cells that are also isolated from a donor of the age of from about 0 to about 5, about 5 to about 10, about 10 to about 15, about 15 to about 20, about 20 to about 25, about 25 to about 30, about 30 to about 35, about 35 to about 40, about 40 to about 45, about 45 to about 50, about 50 to about 55, about 55 to about 60, about 60 to about 65, about 65 to about 70, about 70 to about 75, about 75 to about 80, about 80 to about 85, about 85 to about 90, or about 90 to about 95.

In still other embodiments, one can select the candidate cells showing about 75% or lower, about 70% or lower, about 65% or lower, about 60% or lower, about 55% or lower, about 50% or lower, about 45% or lower, about 40% or lower, about 37% or lower, about 35% or lower, about 32% or lower, about 30% or lower, about 27% or lower, about 25% or lower, about 22% or lower, about 20% or lower, about 17% or lower, about 15% or lower, about 12% or lower, about 10% or lower, about 9% or lower, about 8% or lower, about 7% or lower, about 6% or lower, about 5% or lower, about 4% or lower, about 3% or lower, about 2% or lower, or about 1% or lower in growth rate, and further narrow down to the cells that are also isolated from a donor of the age of from about 0 to about 10, about 10 to about 20, about 20 to about 30, about 30 to about 40, about 40 to about 50, about 50 to about 60, about 60 to about 70, about 70 to about 80, about 80 to about 90, or about 90 to about 100. In addition to the foregoing non-limiting and illustrative examples, any ranges of certain growth rates and any ranges of age can be considered together to select test cells.

### Growth Rate and Donor's Health Condition

In embodiments, one can select the candidate cells showing about 75% or lower, about 70% or lower, about 65% or lower, about 60% or lower, about 55% or lower, about 50% or lower, about 45% or lower, about 40% or lower, about 37% or lower, about 35% or lower, about 32% or lower, about 30% or lower, about 27% or lower, about 25% or lower, about 22% or lower, about 20% or lower, about 17% or lower, about 15% or lower, about 12% or lower, about 10% or lower, about 9% or lower, about 8% or lower, about 7% or lower, about 6% or lower, about 5% or lower, about 4% or lower, about 3% or lower, about 2% or lower, or about 1% or lower in growth rate, and further select the cells that are isolated from a donor who is determined as having a certain clinical condition or a predetermined disease. For example, one can first select candidate cell collections that show, e.g. about 10% or lower in growth rate. Then, upon reviewing the information associated with each candidate cell collection/group, one can further select certain candidate cell collection/group(s) that are isolated from a donor having a certain predetermined disease over the candidate cell collection/group(s) that are isolated from a healthy donor. In embodiments, predetermined disease includes myocardial infarction, central nervous system disorders, stroke, graft-versus-host disease (GVHD), Crohn's disease, spinal cord injury, multiple sclerosis, diabetes, osteoarthritis, osteogenesis imperfecta and other diseases, although not limited thereto. In addition to the foregoing non-limiting and illustrative examples, any ranges of certain growth rates and any health conditions of a donor can be considered together to select test cells.

### Growth Rate and Donor's Gender

In embodiments, one can select the candidate cells showing about 75% or lower, about 70% or lower, about 65% or lower, about 60% or lower, about 55% or lower, about 50% or lower, about 45% or lower, about 40% or lower, about 37% or lower, about 35% or lower, about 32% or lower, about 30% or lower, about 27% or lower, about 25% or lower, about 22% or lower, about 20% or lower, about 17% or lower, about 15% or lower, about 12% or lower, about 10% or lower, about 9% or lower, about 8% or lower, about 7% or lower, about 6% or lower, about 5% or lower, about 4% or lower, about 3% or lower, about 2% or lower, or about 1% or lower in growth rate, and further select the cells that are isolated from a female donor or male donor. In addition to the foregoing non-limiting and illustrative examples, any ranges of certain growth rates can be considered together with gender information to select test cells.

### Growth Rate and Donor's Family Medical History

In embodiments, one can select the candidate cells showing about 75% or lower, about 70% or lower, about 65% or lower, about 60% or lower, about 55% or lower, about 50% or lower, about 45% or lower, about 40% or lower, about 37% or lower, about 35% or lower, about 32% or lower, about 30% or lower, about 27% or lower, about 25% or lower, about 22% or lower, about 20% or lower, about 17% or lower, about 15% or lower, about 12% or lower, about 10% or lower, about 9% or lower, about 8% or lower, about 7% or lower, about 6% or lower, about 5% or lower, about 4% or lower, about 3% or lower, about 2% or lower, or about 1% or lower in growth rate, and further select the cells that are also isolated from a donor having a certain family medical history. For example, one can first select candidate cell collections that show, e.g. about 10% or lower in growth rate.

Then, upon reviewing the information associated with each candidate cell collection/group, one can further select certain candidate cell collection/group(s) that are isolated from a donor linked to a certain predetermined disease in his/her family history over the candidate cell collection/group(s) that are isolated from a donor having no such family medical history. In embodiments, predetermined disease includes myocardial infarction, central nervous system disorders, stroke, graft-versus-host disease (GVHD), Crohn's disease, spinal cord injury, multiple sclerosis, diabetes, osteoarthritis, osteogenesis imperfecta and other diseases, although not limited thereto. In addition to the foregoing non-limiting and illustrative examples, any ranges of certain growth rates and any family medical history of a donor can be considered together to select test cells.

Growth Rate and Source Tissue

In embodiments, one can select the candidate cells showing about 75% or lower, about 70% or lower, about 65% or lower, about 60% or lower, about 55% or lower, about 50% or lower, about 45% or lower, about 40% or lower, about 37% or lower, about 35% or lower, about 32% or lower, about 30% or lower, about 27% or lower, about 25% or lower, about 22% or lower, about 20% or lower, about 17% or lower, about 15% or lower, about 12% or lower, about 10% or lower, about 9% or lower, about 8% or lower, about 7% or lower, about 6% or lower, about 5% or lower, about 4% or lower, about 3% or lower, about 2% or lower, or about 1% or lower in growth rate, and further select the cells that are isolated from a certain tissue. For example, one can first select candidate cell collections that show, e.g. about 10% or lower in growth rate.

Then, upon reviewing the information associated with each candidate cell collection/group, one can further select certain candidate cell collection/group(s) that are isolated from a certain predetermined type of tissues over the candidate cell collection/group(s) that are isolated from other types of tissues. In embodiments, predetermined types of tissues includes brain, bone marrow, blood vessels, skeletal muscle, skin, teeth, heart, gut, liver, adipose tissue, cord blood, placenta, and other organs and tissues, although not limited thereto. In addition to the foregoing non-limiting and illustrative examples, any ranges of certain growth rates and any types of source tissue can be considered together to select test cells.

### Growth Rate and Previous In Vitro Culture

In embodiments, one can select the candidate cells showing about 75% or lower, about 70% or lower, about 65% or lower, about 60% or lower, about 55% or lower, about 50% or lower, about 45% or lower, about 40% or lower, about 37% or lower, about 35% or lower, about 32% or lower, about 30% or lower, about 27% or lower, about 25% or lower, about 22% or lower, about 20% or lower, about 17% or lower, about 15% or lower, about 12% or lower, about 10% or lower, about 9% or lower, about 8% or lower, about 7% or lower, about 6% or lower, about 5% or lower, about 4% or lower, about 3% or lower, about 2% or lower, or about 1% or lower in growth rate, and further select the cells that are not previously cultured in vitro after being isolated from a donor. Alternatively, the cells that are previously cultured in vitro can be further selected. In addition to the foregoing non-limiting and illustrative examples, any ranges of certain growth rates and any previous culture history can be considered together to select test cells.

### Growth Rate, Age and Health Condition

In embodiments, one can select a test cell collection/group(s) from a number of candidate cell collections based on their growth rate and more than one additional piece of information relating to the candidate cells. For example, the growth rate, donor's age and donor's health condition are considered together to rank and/or select test cell collections.

In embodiments, one can select the candidate cells showing about 75% or lower, about 70% or lower, about 65% or lower, about 60% or lower, about 55% or lower, about 50% or lower, about 45% or lower, about 40% or lower, about 37% or lower, about 35% or lower, about 32% or lower, about 30% or lower, about 27% or lower, about 25% or lower, about 22% or lower, about 20% or lower, about 17% or lower, about 15% or lower, about 12% or lower, about 10% or lower, about 9% or lower, about 8% or lower, about 7% or lower, about 6% or lower, about 5% or lower, about 4% or lower, about 3% or lower, about 2% or lower, or about 1% or lower in growth rate, and further narrow down to the cells that are isolated from a donor of the age of 20 or higher, 21 or higher, 22 or higher, 23 or higher, 24 or higher, 25 or higher, 26 or higher, 27 or higher, 28 or higher, 29 or higher, 30 or higher, 31 or higher, 32 or higher, 33 or higher, 34 or higher, 35 or higher, 36 or higher, 37 or higher, 38 or higher, 39 or higher, 40 or higher, 41 or higher, 42 or higher, 43 or higher, 44 or higher, 55 or higher, 46 or higher, 47 or higher, 48 or higher, 49 or higher, 50 or higher, 51 or higher, 52 or higher, 53 or higher, 54 or higher, 55 or higher, 56 or higher, 57 or higher, 58 or higher, 59 or higher, 60 or higher, 61 or higher, 62 or higher, 63 or higher, 64 or higher, 65 or higher, 66 or higher, 67 or higher, 68 or higher, 69 or higher, 70 or higher, 71 or higher, 72 or higher, 73 or higher, 74 or higher, 75 or higher, 76 or higher, 77 or higher, 78 or higher, 79 or higher, 80 or higher, 81 or higher, 82 or higher, 83 or higher, 84 or higher, 85 or higher, 86 or higher, 87 or higher, 88 or higher, 89 or higher, 90 or higher, 91 or higher, 92 or higher, 93 or higher, 94 or higher, or 95 or higher and further the cells that were isolated from the donor having a certain predetermined clinical condition or disease. For example, one can first select candidate cell collections that show, e.g. about 15% or lower in growth rate. Then, upon reviewing the information associated with each candidate cell collection/group, one can further select certain candidate cell collection/group(s) that are isolated from a donor of, e.g. the age of 45 or higher who has cancer over other candidate cell collection/group(s) that are isolated from a healthy donor.

In embodiments, one can select the candidate cells showing about 75% or lower, about 70% or lower, about 65% or lower, about 60% or lower, about 55% or lower, about 50% or lower, about 45% or lower, about 40% or lower, about 37% or lower, about 35% or lower, about 32% or lower, about 30% or lower, about 27% or lower, about 25% or lower, about 22% or lower, about 20% or lower, about 17% or lower, about 15% or lower, about 12% or lower, about 10% or lower, about 9% or lower, about 8% or lower, about 7% or lower, about 6% or lower, about 5% or lower, about 4% or lower, about 3% or lower, about 2% or lower, or about 1% or lower in growth rate, and further narrow down to the cells that are also isolated from a donor of the age of 20 or lower, 21 or lower, 22 or lower, 23 or lower, 24 or lower, 25 or lower, 26 or lower, 27 or lower, 28 or lower, 29 or lower, 30 or lower, 31 or lower, 32 or lower, 33 or lower, 34 or lower, 35 or lower, 36 or lower, 37 or lower, 38 or lower, 39 or lower, 40 or lower, 41 or lower, 42 or lower, 43 or lower, 44 or lower, 55 or lower, 46 or lower, 47 or lower, 48 or lower, 49 or lower, 50 or lower, 51 or lower, 52 or lower, 53 or lower, 54 or lower, 55 or lower, 56 or lower, 57 or lower, 58 or lower, 59 or lower, 60 or lower, 61 or lower, 62 or lower, 63 or lower, 64 or lower, 65 or lower, 66 or lower, 67 or lower, 68 or lower, 69 or lower, 70 or lower, 71 or lower, 72 or lower, 73 or lower, 74 or lower, 75 or lower, 76 or lower, 77 or lower, 78 or lower, 79 or lower, 80 or lower, 81 or lower, 82 or lower, 83 or lower, 84 or lower, 85 or lower, 86 or lower, 87 or lower, 88 or lower, 89 or lower, 90 or lower, 91 or lower, 92 or lower, 93 or lower, 94 or lower, or 95 or lower who has a certain predetermined clinical condition or disease. In embodiments, predetermined disease includes myocardial infarction, central nervous system disorders, stroke, graft-versus-host disease (GVHD), Crohn's disease, spinal cord injury, multiple sclerosis, diabetes, osteoarthritis, osteogenesis imperfecta and other diseases, although not limited thereto. For example, one can first select candidate cell collections that show, e.g. about 15% or lower in growth rate. Then, upon reviewing the information associated with each candidate cell collection/group, one can further select certain candidate cell collection/group(s) that are isolated from a donor of, e.g. the age of 45 or lower, who is diabetic over other candidate cell collection/group(s) that are isolated from a healthy donor.

In embodiments, one can select the candidate cells showing about 75% or lower, about 70% or lower, about 65% or lower, about 60% or lower, about 55% or lower, about 50% or lower, about 45% or lower, about 40% or lower, about 37% or lower, about 35% or lower, about 32% or lower, about 30% or lower, about 27% or lower, about 25% or lower, about 22% or lower, about 20% or lower, about 17% or lower, about 15% or lower, about 12% or lower, about 10% or lower, about 9% or lower, about 8% or lower, about 7% or lower, about 6% or lower, about 5% or lower, about 4% or lower, about 3% or lower, about 2% or lower, or about 1% or lower in growth rate, and further narrow down to the cells that are also isolated from a donor of the age of from about 0 to about 5, about 5 to about 10, about 10 to about 15, about 15 to about 20, about 20 to about 25, about 25 to about 30, about 30 to about 35, about 35 to about 40, about 40 to about 45, about 45 to about 50, about 50 to about 55, about 55 to about 60, about 60 to about 65, about 65 to about 70, about 70 to about 75, about 75 to about 80, about 80 to about 85, about 85 to about 90, or about 90 to about 95. In embodiments, one can select the candidate cells showing about 75% or lower, about 70% or lower, about 65% or lower, about 60% or lower, about 55% or lower, about 50% or lower, about 45% or lower, about 40% or lower, about 37% or lower, about 35% or lower, about 32% or lower, about 30% or lower, about 27% or lower, about 25% or lower, about 22% or lower, about 20% or lower, about 17% or lower, about 15% or lower, about 12% or lower, about 10% or lower, about 9% or lower, about 8% or lower, about 7% or lower, about 6% or lower, about 5% or lower, about 4% or lower, about 3% or lower, about 2% or lower, or about 1% or lower in growth rate, and further narrow down to the cells that are isolated from a donor of the age of from about 0 to about 10, about 10 to about 20, about 20 to about 30, about 30 to about 40, about 40 to about 50, about 50 to about 60, about 60 to about 70, about 70 to about 80, about 80 to about 90, or about 90 to about 100 who has a certain predetermined clinical condition or disease.

In embodiments, predetermined disease includes myocardial infarction, central nervous system disorders, stroke, graft-versus-host disease (GVHD), Crohn's disease, spinal cord injury, multiple sclerosis, diabetes, osteoarthritis, osteogenesis imperfecta and other diseases, although not limited thereto. For example, one can first select candidate cell collections that show, e.g. about 15% or lower in growth rate. Then, upon reviewing the information associated with each candidate cell collection/group, one can further select certain candidate cell collection/group(s) that are isolated from a donor of, e.g. the age of from about 40 to about 50 having, for example, myocardial infarction, central nervous system disorders, stroke, graft-versus-host disease (GVHD), Crohn's disease, spinal cord injury, multiple sclerosis, diabetes, osteoarthritis, osteogenesis imperfecta or other disease over other candidate cell collection/group(s) that are isolated from a healthy donor with different an age group. Besides any ranges of certain growth rates, any ranges of age, and any clinical condition or disease information can be considered together to select test cells.

### Consideration of Multiple Pieces of Information

Also, two or more pieces of information selected from the group consisting of the donor's age, the donor's weight, the donor's gender, the donor's health condition, the donor's previous medical history, the donor's family medical history, source tissue, the condition of the source tissue, and in vitro culture history can be considered along with the growth rate. For instance, growth rate, age, donor's current health condition and family medical history may be considered together to rank or select test cells. In another example, growth rate, donor's current health condition, source tissue and the condition of the source tissue may be considered together. Various other combinations and permutations of the above listed information and growth rate can be possible.

### Selection of Test Cells

The candidate cells selected from the foregoing considerations of the growth rate data and the associated information would be defined as the cells having a relatively lower growth rate and also being associated with the factors that may negatively affect the cell growth. Due to these characteristics, the selected cells are more sensitive on the quality of a growth medium and would not grow well if the culture medium is not optimized. As said above, the FBS quality is important to determine the quality of medium. Accordingly, the selected cells having high sensitivity to the culture medium can be used as test cells for identifying FBS that is suitable for culturing other sensitive cells such as primary stem cells.

### Use of Selected Test Cells

In embodiments, after selecting test cell collections, the selected test cell collections are used for selecting FBS batches to provide a cell culture media for primary stem cell culture. The test cell collections are used to test and select FBS batches that are suitable for culturing primary stem cells that are isolated from an autologous or allogeneic source. Also, the test cell collections can be used to establish optimized culturing conditions for primary stem cells. The primary stem cells are often sensitive to, e.g. changes in pH, concentrations of ingredients in cell culture medium, concentration of gases present in the medium, temperature, light, and many other environmental factors. Accordingly, one can culture the selected test cells in different culturing conditions, e.g. different pH, different concentrations of ingredients, different concentrations of gases present in the medium, different temperatures, different light intensity, and many other environmental factors, and identify the conditions from which the test cells grow well. The identified conditions can be applicable to the primary stem cell culture.

### Selection of Effective FBS Batches

In order to successfully operate cell therapy, it is important to establish an optimal culture system in which the cells will grow and expand while retaining their biological characteristics. Therefore, upon testing new batches of FBS, one can select certain one of them that would allow the cells to efficiently expand and grow while maintaining their biological characteristics. In embodiments, cell growth/health and biological characteristics can be measured by the following methods. These methods can be conducted for the selection of effective FBS batches.

First, the morphology, growth rate, and adherence characteristics of cells are monitored while culturing the cells in a culture medium containing FBS. For example, when mesenchymal stem cells are cultured in a typical DMEM basal media containing 10% FBS, they are known to have a spindle-shaped fibroblast-like morphology and be adherent to the surface of a cell culture dish. Therefore, when a new batch of FBS is tested, one can monitor if the test cells grown in a test medium containing the new batch of FBS will still have (1) a spindle-shaped fibroblast-like morphology and (2) adherence characteristics. Also, the growth rate of the test cell collections can be measured and compared between the media containing the old FBS and a new batch of FBS. If the growth rate of the test cells grown in the new batch of FBS is significantly lower than that of the test cells grown in the old medium, the new batch of FBS would not be selected.

Second, individual stem cells are known to have a specific immunophenotype profile. For example, mesenchymal stem cells are known to express certain surface markers, such as CD63, CD105, CD166, CD54, CD13, CD44, and CD73. On the other hand, mesenchymal stem cells are known to be negative to certain hematopoietic markers, such as CD34, CD45, CD14, CD116, CD19, and CD133. Therefore, one or more surface marker expression profile would be helpful to monitor for determining if the culture cells are maintaining the mesenchymal stem cell status.

Third, individual stem cells are known to have a capability to differentiate into many different fates of tissues. For example, mesenchymal stem cells are known to differentiate into bone, cartilage, adipose tissue, and neuronal tissue under certain induction conditions. Accordingly, in order to confirm that the cultured mesenchymal stem cells would maintain such multipotency, one can provide a specific induction condition to the cultured cells and monitor if the induced cells would differentiate to the intended fate of tissues.

In some embodiments, the following can be considered as the criteria for defining mesenchymal stem cells:
Mesenchymal stem cells are plastic-adherent, i.e. adhering to the surface of a cell culture device
Expression profiles of certain surface markers can be measured. For example, if more than about 80%, about 81%, about 82%, about 83%, about 84%, about 85%, about 86%, about 87%, about 88%, about 89%, about 90%, about 91%, about 92%, about 93%, about 94%, about 95%, about, 96%, about 97%, about 98%, or about 99% of the total population of the cultured cells express at one selected from the group consisting of CD105, CD73, and CD90, this may indicate that the cultured cells maintain the characteristics of mesenchymal stem cells. Also, in another example, if less than about 10%, about 9%, about 8%, about 7%, about 6%, about 5%, about 4%, about 3%, about 2%, or about 1% of the total population of the cultured cells express at least one selected from the group consisting of CD45, CD34, CD14 or CD11B, CD79a or CD19, and HLA-DR, this may indicate that the cultured cells maintain the characteristics of mesenchymal stem cells.
Under a certain induction condition, mesenchymal stem cells would be able to differentiate into osteoblast, adipocyte, and/or chondroblast.

### Criteria Evaluating Cultured Mesenchymal Stem Cells (MSC)

In order to successfully operate cell therapy using stem cells, e.g. mesenchymal stem cells (MSC), it is essential to have an optimized cell culture in which MSC would stably grow and divide while maintaining their biological characteristics. The culture medium suitable for MSC will be able to allow stable growth of MSC without losing their biological characteristics. Accordingly, in some embodiments, one can use the following selection criteria when selecting a batch of FBS that is suitable for culturing primary stem cells for cell therapy.

TABLE 1
Examples of criteria evaluating cultured mesenchymal stem cells (MSC)

| Evaluation criteria | | Evaluation test |
|---|---|---|
| Growth rate | | Variable (further discussed) |
| Cell morphology | | Plastic-adherent fibroblast-like morphology |
| Immunophenotype | Positive surface markers | About 80%, about 81%, about 82%, about 83%, about |
| | (CD105, CD73) | 84%, about 85%, about 86%, about 87%, about 88%, |
| | | about 89%, about 90%, about 91%, about 92%, about |
| | | 93%, about 94%, about 95%, about, 96%, about 97%, |
| | | about 98%, about 99% or more of the cultured cells |
| | | would express positive surface makers. |
| | Negative surface | About 10%, about 9%, about 8%, about 7%, about 6%, |
| | markers (CD14, CD34, | about 5%, about 4%, about 3%, about 2%, or about 1% |
| | CD45) | of the cultured cells would express negative surface |
| | | markers. |
| Differentiation | Osteoblast and | Staining of markers associated with osteoblast and/or |
| potency | adipocyte | adipocyte will be positive. |
| | Cardiomyocyte | Troponin T-positive staining will be detected. |

In addition to the foregoing criteria, one can further apply additional criteria such as capability to migrate toward damaged tissues and secretion of bioactive agents (e.g. growth factors, cytokines, and others) to evaluate mesenchymal stem cells for further selecting suitable FBS. Further, more surface markers can be measured to evaluate mesenchymal stem cell status.

Certain specific examples are provided to help understanding of some aspects of the invention and do not intend to limit the scope of the invention.

### Example 1

### Preparation of a Plurality of Candidate Cell Collections

Total 133 donors were recruited and a group/collection of mononuclear cells was isolated from the bone marrow of each donor. In addition, information including age, gender, weight, current disease/clinical condition of the donor as well as the amount of isolated bone marrow tissues was collected for each group/collection of the isolated cells. Each group/collection of the isolated cells was used as a candidate cell collection/group in the experiment conducted for selecting a test cell collection/group(s).

In one embodiment, a group/collection of the mononuclear cells isolated from the donor was used as a collection/group of candidate cells. In another embodiment, a collection/group of mesenchymal stem cells (MSC) which were isolated from the initial culture of mononuclear cells was used as a collection/group of candidate cells.

### Example 2

### Preparation of Database

Various types of information, which were collected for each group/collection of the candidate cells, were processed and incorporated into a database. This database was processed to run statistical analyses, e.g. selecting a certain group(s)/collection(s) of the candidate cells which were isolated from a certain age group, a certain gender, a certain weight range and/or having a certain disease/clinical condition. Further, the database used to rank the candidate cells based on certain criteria. Using the database, one ranks the candidate cells from the highest to the lowest based on the age of the donor. Accordingly, the group/collection of the candidate cells isolated from the oldest donor would be ranked the highest whereas another group/collection of the candidate cells isolated from the youngest donor would be ranked the lowest. The ranking criteria are the combination of two or more types of information, e.g. age and disease/clinical condition. Therefore, the group/collection of candidate cells isolated from an aged donor having a certain disease would be ranked higher than the group/collection of candidate cells isolated from a younger donor having no disease. The criteria for ranking the candidate cells can comprise any possible combinations of the collected types of information. As described later, a growth rate of each of the candidate cell collections was measured and the data of this growth rate were processed together with other types of information so as to rank the candidate cells and to determine the test cell collections.

The following table shows the set of information incorporated into the database.

TABLE 2
Set of collected information

| | | | | | Amount of |
|---|---|---|---|---|---|
| | | | | Disease/ | aspirated |
| Donor | | | | Clinical | bone marrow |
| ID. No. | Age | Gender | Weight | condition | (mL) |
| 1 | 76 | M | 70 | Myocardial infarction | 20 |
| 2 | 87 | M | 70 | Myocardial infarction | 20 |
| 3 | 73 | F | 50 | Myocardial infarction | 29.5 |
| 4 | 70 | F | 70 | Myocardial infarction | 30 |
| 5 | 60 | F | 50 | Myocardial infarction | 30 |
| 6 | 53 | M | 50 | Myocardial infarction | 30 |
| 7 | 47 | M | 70 | Myocardial infarction | 30 |
| 8 | 74 | F | 70 | Myocardial infarction | 30 |
| 9 | 82 | F | 50 | Myocardial infarction | 36.5 |
| 10 | 82 | M | 50 | Myocardial infarction | 36.5 |
| 11 | 57 | M | 80 | Myocardial infarction | 30 |
| 12 | 69 | M | 70 | Myocardial infarction | 30 |
| 13 | 63 | F | 70 | Myocardial infarction | 36 |
| 14 | 57 | M | 70 | Myocardial infarction | 37 |
| 15 | 61 | F | 50 | Myocardial infarction | 30 |
| 16 | 76 | M | 70 | Myocardial infarction | 29 |
| 17 | 76 | F | 70 | Myocardial infarction | 40 |
| 18 | 67 | M | 70 | Myocardial infarction | 40 |
| 19 | 59 | M | 70 | Myocardial infarction | 40 |
| 20 | 75 | F | 70 | Myocardial infarction | 30 |
| 21 | 72 | M | 70 | Myocardial infarction | 40 |
| 22 | 73 | M | 70 | Myocardial infarction | 30 |
| 23 | 63 | F | 50 | Myocardial infarction | 40 |
| 24 | 76 | M | 50 | Myocardial infarction | 30 |
| 25 | 41 | M | 80 | Myocardial infarction | 25.5 |
| 26 | 69 | M | 70 | Myocardial infarction | 27.5 |
| 27 | 65 | M | 70 | Myocardial infarction | 24.5 |
| 28 | 71 | M | 50 | Myocardial infarction | 30 |
| 29 | 66 | F | 50 | Myocardial infarction | 30 |
| 30 | 72 | M | 80 | Myocardial infarction | 29.5 |
| 31 | 66 | F | 50 | Myocardial infarction | 29.5 |
| 32 | 70 | M | 70 | Myocardial infarction | 30.5 |
| 33 | 51 | M | 80 | Myocardial infarction | 36.5 |
| 34 | 52 | F | 50 | Myocardial infarction | 23.5 |
| 35 | 74 | F | 70 | Myocardial infarction | 30 |
| 36 | 57 | M | 70 | Myocardial infarction | 30 |
| 37 | 49 | M | 50 | Myocardial infarction | 30 |
| 38 | 57 | M | 80 | Myocardial infarction | 30 |
| 39 | 63 | M | 70 | Myocardial infarction | 41 |
| 40 | 66 | F | 50 | Myocardial infarction | 30 |
| 41 | 62 | M | 80 | Myocardial infarction | 30 |
| 42 | 43 | M | 50 | Myocardial infarction | 30 |
| 43 | 69 | F | 70 | Myocardial infarction | 24 |
| 44 | 72 | M | 50 | Myocardial infarction | 30 |
| 45 | 56 | M | 80 | Myocardial infarction | 25 |
| 46 | 67 | F | 70 | Myocardial infarction | 12.5 |
| 47 | 33 | F | 70 | Myocardial infarction | 24 |
| 48 | 73 | F | 50 | Myocardial infarction | 30 |
| 49 | 60 | M | 70 | Myocardial infarction | 30 |
| 50 | 58 | F | 70 | Myocardial infarction | 22.5 |
| 51 | 75 | F | 50 | Myocardial infarction | 5.4 |
| 52 | 70 | M | 70 | Myocardial infarction | 30 |
| 53 | 78 | F | 50 | Myocardial infarction | 22.5 |
| 54 | 66 | M | 50 | Myocardial infarction | 30 |
| 55 | 49 | M | 70 | Myocardial infarction | 16.5 |
| 56 | 60 | M | 70 | Myocardial infarction | 22.5 |
| 57 | 69 | F | 70 | Myocardial infarction | 30 |
| 58 | 64 | M | 70 | Myocardial infarction | 30 |
| 59 | 72 | M | 70 | Myocardial infarction | 22.5 |
| 60 | 68 | F | 70 | Myocardial infarction | 30 |
| 61 | 74 | F | 70 | Myocardial infarction | 21.5 |
| 62 | 62 | F | 50 | Myocardial infarction | 30 |
| 63 | 49 | F | 70 | Myocardial infarction | 21.5 |
| 64 | 50 | F | 80 | Myocardial infarction | 29.5 |
| 65 | 57 | F | 50 | Myocardial infarction | 19.5 |
| 66 | 30 | M | 70 | Myocardial infarction | 24 |
| 67 | 76 | M | 70 | Myocardial infarction | 27 |
| 68 | 59 | M | 70 | Myocardial infarction | 28 |
| 69 | 44 | F | 50 | Myocardial infarction | 11 |
| 70 | 75 | M | 70 | Myocardial infarction | 19.5 |
| 71 | 52 | M | 80 | Myocardial infarction | 30 |
| 72 | 64 | M | 80 | Myocardial infarction | 21 |
| 73 | 64 | M | 70 | Myocardial infarction | 27.5 |
| 74 | 37 | M | 53.5 | Liver cirrhosis | 27.0 |
| 75 | 52 | M | 53 | Liver cirrhosis | 20.0 |
| 76 | 60 | M | 51.45 | Liver cirrhosis | 21.0 |
| 77 | 53 | M | 54.85 | Liver cirrhosis | 23.0 |
| 78 | 58 | M | 54.7 | Liver cirrhosis | 28.0 |
| 79 | 58 | M | 57.7 | Liver cirrhosis | 28.0 |
| 80 | 53 | M | 83.7 | Liver cirrhosis | 30.0 |
| 81 | 48 | M | 47.2 | Liver cirrhosis | 14.7 |
| 82 | 49 | M | 58.7 | Liver cirrhosis | 25.0 |
| 83 | 50 | F | 47.8 | Liver cirrhosis | 17.5 |
| 84 | 40 | M | 66.35 | Liver cirrhosis | 29.0 |
| 85 | 40 | M | 76 | Liver cirrhosis | 23.0 |
| 86 | 48 | M | 67 | Ischemic stroke | 16.2 |
| 87 | 73 | M | 55 | Ischemic stroke | 20.0 |
| 88 | 66 | F | 55 | Ischemic stroke | 25.0 |
| 89 | 56 | M | 67 | Ischemic stroke | 24.0 |
| 90 | 69 | M | 65 | Ischemic stroke | 14.2 |
| 91 | 31 | M | 93 | Spinal cord injury | 17.0 |
| 92 | 32 | M | 63 | Spinal cord injury | 18.0 |
| 93 | 16 | M | 43 | Spinal cord injury | 17.0 |
| 94 | 29 | M | 65 | Spinal cord injury | 15.0 |
| 95 | 44 | M | 65 | Spinal cord injury | 14.5 |
| 96 | 18 | M | 66 | Spinal cord injury | 17.5 |
| 97 | 38 | M | 67 | Spinal cord injury | 12.0 |
| 98 | 64 | M | 55 | Spinal cord injury | 14.0 |
| 99 | 26 | M | 63 | Spinal cord injury | 19.5 |
| 100 | 40 | M | 76 | Spinal cord injury | 19.5 |
| 101 | 57 | M | 69 | Spinal cord injury | 19.5 |
| 102 | 42 | M | 57 | Spinal cord injury | 19.0 |
| 103 | 43 | M | 70 | Spinal cord injury | 19.0 |
| 104 | 21 | M | 80 | Spinal cord injury | 19.5 |
| 105 | 32 | M | 50 | Spinal cord injury | 18.0 |
| 106 | 45 | M | 62 | Spinal cord injury | 19.5 |
| 107 | 54 | M | 70.3 | Myocardial infarction | 40 |
| 108 | 54 | M | 65.6 | Myocardial infarction | 50 |
| 109 | 43 | M | 87 | Myocardial infarction | 42 |
| 110 | 46 | M | 67.3 | Myocardial infarction | 15 |
| 111 | 51 | M | 82 | Myocardial infarction | 18 |
| 112 | 70 | F | 67.75 | Myocardial infarction | 17 |
| 113 | 56 | M | 63 | Myocardial infarction | 23 |
| 114 | 65 | M | 69 | Myocardial infarction | 18 |
| 115 | 58 | F | 64 | Myocardial infarction | 16.5 |
| 116 | 46 | M | 78 | Myocardial infarction | 19 |
| 117 | 27 | M | 98.9 | Myocardial infarction | 21 |
| 118 | 69 | M | 70.2 | Myocardial infarction | 23.5 |
| 119 | 46 | M | 71 | Myocardial infarction | 23.5 |
| 120 | 48 | M | 84 | Myocardial infarction | 20.8 |
| 121 | 54 | M | 57.7 | Myocardial infarction | 23 |
| 122 | 66 | F | 59.4 | Myocardial infarction | 19 |
| 123 | 44 | M | 81 | Myocardial infarction | 30.5 |
| 124 | 69 | M | 70.65 | Myocardial infarction | 23 |
| 125 | 66 | M | 68.9 | Myocardial infarction | 15 |
| 126 | 63 | F | 66 | Myocardial infarction | 8 |
| 127 | 50 | M | 75.8 | Myocardial infarction | 22 |
| 128 | 52 | M | 63.7 | Myocardial infarction | 20 |
| 129 | 62 | M | 61.9 | Myocardial infarction | 20 |
| 130 | 52 | M | 70 | Myocardial infarction | 12 |
| 131 | 41 | M | 59 | Myocardial infarction | 17 |
| 132 | 63 | M | 68.4 | Myocardial infarction | 17 |
| 133 | 44 | M | 65 | Myocardial infarction | 21.5 |

### Example 3

### Measurement of Cell Growth Rate

The isolated 133 collections of candidate cells were cultured in the existing culture media. A mixture of mononuclear cells was isolated from an individual donor. In some embodiments, the isolated mononuclear cells without being cryopreserved were used in the test. Alternatively, the isolated mononuclear cells were cryopreserved before testing. In such a case, the frozen cells were thawed before proceeding to the test.

When the mononuclear cells were ready for the test, an average number of 10.58×10⁷ cells from each collection/group were seeded in a flask (e.g. T75 or T175 flask) having 20 mL of cell culture medium at day 0 and cultured under 37° C., 5% of humidity for about 12 days. This initial culture of mononuclear cells is defined as P0 stage. The media was prepared with DMEM basal media and 10% FBS was added thereto. In addition, antibiotics (e.g. gentamycin, streptomycin, or penicillin) were added. After P0 stage, i.e. at about day 13, the cultured cells, which were largely MSC, were transferred to a fresh medium of 30 mL advancing to a next passage (P1). The cells were continuously cultured, and in general the cultured cells were counted and transferred to a fresh medium at every 3-5 days. Cell counting was done by trypan blue staining. More particularly, a diluted 10 μL cell culture solution was mixed with 10 μL of trypan blue staining solution and the mixture was applied to two separate counting chamber of haematocytometer. Each candidate cell collection/group was undergone for total five passages. Cellular morphology, adherence, cell density and growth rate were monitored.

From the growth rate of each candidate cell collection/group for individual passage, the following results were obtained.

TABLE 3
Growth Rate Comparison

| | | | | | | | Average | Total |
|---|---|---|---|---|---|---|---|---|
| | | | | | | | growth | growth |
| | P0 | P1 | P2 | P3 | P4 | P5 | rate | rate |
| Growth rate of the | 2.19 | 1.41 | 1.45 | 1.79 | 1.17 | 1.52 | 1.58 | 14.28 |
| candidate showing | | | | | | | | |
| the lowest growth | | | | | | | | |
| rate (n = 1) | | | | | | | | |
| Growth rate of the | 6.17 | 4.76 | 6.28 | 2.10 | 2.10 | 1.90 | 3.88 | 1545.91 |
| candidate showing | | | | | | | | |
| the highest growth | | | | | | | | |
| rate (n = 1) | | | | | | | | |
| Average growth rate | 1.71 | 1.94 | 1.91 | 2.00 | 1.66 | 1.67 | 1.81 | 33.77 |
| for the cells showing | | | | | | | | |
| 10% or less of total | | | | | | | | |
| growth rate (n = 13) | | | | | | | | |
| Average growth rate | 1.84 | 2.03 | 1.99 | 1.97 | 1.7 | 1.73 | 1.87 | 42.32 |
| for the cells showing | | | | | | | | |
| 20% or less of total | | | | | | | | |
| growth rate (n = 26) | | | | | | | | |
| Average growth rate | 2.02 | 2.08 | 2.09 | 1.96 | 1.85 | 1.74 | 1.95 | 54.91 |
| for the cells showing | | | | | | | | |
| 30% or less of total | | | | | | | | |
| growth rate (n = 39) | | | | | | | | |
| Average growth rate | 2.18 | 2.16 | 2.07 | 2.01 | 1.89 | 1.77 | 2.01 | 64.82 |
| for the cells showing | | | | | | | | |
| 40% or less of total | | | | | | | | |
| growth rate (n = 53) | | | | | | | | |
| Average growth rate | 2.73 | 2.58 | 2.59 | 2.44 | 2.24 | 1.84 | 2.40 | 229.69 |
| of total 133 | | | | | | | | |
| candidates | | | | | | | | |

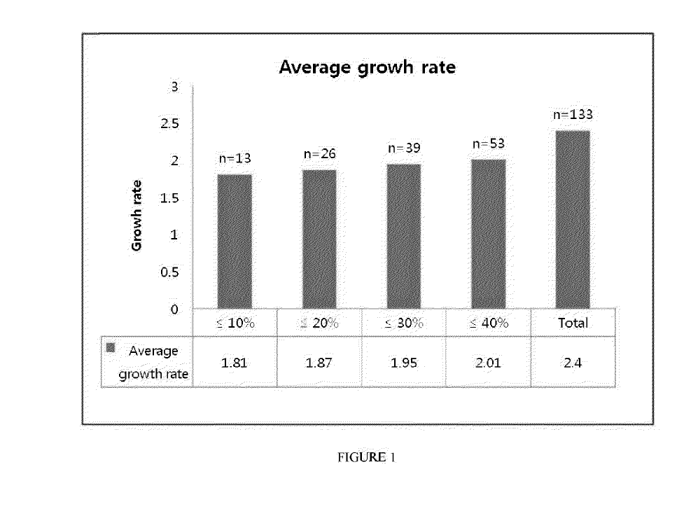

As seen from the above Table 2, the growth rates between candidate cells were significantly different. The total growth rate of the candidate cell collection/group showing the highest growth rate was 1545.91 whereas the total growth rate of the candidate cell collection/group showing the least growth rate was 14.28, the difference between these two being more than 100-fold. In addition, the candidate cell collections showing 10% or lower growth rate had an average of 33.77 which is significantly lower than the average growth rate of all 133 candidate cell collections, i.e. 226.69. Further, even for those showing 40% or lower growth rate had an average growth rate of 64.82 which was much lower than the total average growth rate of 226.69. The average growth rate is illustrated in FIG. 1.

The tested 133 cells underwent the environmental change, i.e. from in vivo to in vitro. Some cells grew relatively better than some other cells. This means that some cells were less sensitive to the environmental changes and the artificial factors present in the culturing condition. On the other hand, there were some other cells that were more sensitive to the environmental changes and such that their growth rates were more significantly affected than the less sensitive cells when cultured in vitro. The cells having lower growth rates would be more restrictive in terms of selecting a habitable artificial environment (i.e. cell culture condition), and therefore if the efficacy of the culture medium were not good enough, they could have difficulty in maintaining their growth. The sensitivity to the culture medium and necessity of high efficacy medium were relatively high in the lower-growing cells than the higher-growing cells.

As said earlier, the sensitivity to the culture medium would be quite divergent in different primary stem cells. Especially, when an autologous therapy where a donor of the primary stem cells is a patient is concerned, the sensitivity of such cells to environmental changes could be higher than other cells, e.g. those isolated from a healthy non-patient donor. Such autologous stem cells may not grow well in vitro unless the culture medium is in an optimized condition, and this can lead to an unsuccessful cell therapy. The test cells selected by the method of the present invention would be particularly useful as they also have a high sensitivity to a culture medium. Therefore, the test cell collection/group(s) can be representative of certain primary stem cells that are sensitive to a culture medium. Accordingly, the selected test cell collections can be used to test and optimize cell culture medium for such primary stem cells.

### Example 4

### Further Selection of Candidate Cells Upon Combinatorial Consideration of the Growth Rate and Information Associated with a Donor

In addition to the growth rate comparison, information that is associated with each cell collection/group can be helpful in selecting test cell collections. As described above, various types of information which may be relevant to the characteristics of the isolated cells were collected. Information of the donor's age, gender, weight, and current disease was collected per each candidate cell collection/group.

### Example 4.1

### Age Information

The following table shows the distribution of age groups in total 133 candidate cell collections.

TABLE 4
Age groups in the donors of the 133 candidate cell collections

| | Age groups (years) | | | | | | | |
|---|---|---|---|---|---|---|---|---|
| | 19 or | | | | | | | 80 or |
| | less | 20-29 | 30-39 | 40-49 | 50-59 | 60-69 | 70-79 | more |
| Number of | 2 | 4 | 7 | 25 | 31 | 36 | 25 | 3 |
| donors | | | | | | | | |

### Example 4.2

### Gender Information

The following table shows the gender distribution among the donors of the 133 candidate collections.

TABLE 5
Gender distribution of the donors of the 133 candidate cell collections

| Gender | Male | Female |
|---|---|---|
| Number of donors | 97 | 36 |

### Example 4.3

### Weight Information

The following table shows the weight distribution among the donors of the 133 candidate collections.

TABLE 6
Weight distribution of the donors of
the 133 candidate cell collections

| | Weight range | | | | | |
|---|---|---|---|---|---|---|
| | 50 kg | 50 kg to | 60 kg to | 70 kg to | 80 kg to | 90 kg |
| | or | less than | less than | less than | less than | or |
| | less | 60 kg | 70 kg | 80 kg | 90 kg | higher |
| Number | 3 | 38 | 24 | 50 | 16 | 2 |
| of | | | | | | |
| donors | | | | | | |

### Example 4.4

### Health Information

The following table shows the diseases associated with the donors of the 133 candidate collections

TABLE 7
Diseases associated with the donors
of the 133 candidate cell collections

| | Myocardial | Ischemic | Spinal cord | Liver |
|---|---|---|---|---|
| Disease | infarction | stroke | injury | cirrhosis |
| Number of | 80 | 5 | 16 | 12 |
| donors | | | | |

### Example 4.5

### Selection of Test Cells

Among the candidate cell collections which show about 10% or lower growth rates, cell collections isolated from a donor of the age of 80 or higher are further selected.

### Example 4.6

### Selection of Test Cells

Among the candidate cell collections which show about 10% or lower growth rates, cell collections isolated from a donor of the age of 70 or higher are further selected.

### Example 4.7

### Selection of Test Cells

Among the candidate cell collections which show about 10% or lower growth rates, cell collections isolated from a donor of the age of 60 or higher are further selected.

### Example 4.8

### Selection of Test Cells

Among the candidate cell collections which show about 10% or lower growth rates, cell collections isolated from a donor of the age of 50 or higher are further selected.

### Example 4.9

### Selection of Test Cells

Among the candidate cell collections which show about 10% or lower growth rates, cell collections isolated from a male donor are further selected.

### Example 4.10

### Selection of Test Cells

Among the candidate cell collections which show about 10% or lower growth rates, the cell collections isolated from a female donor are further selected.

### Example 4.11

### Selection of Test Cells

Among the candidate cell collections which show about 10% or lower growth rates, cell collections isolated from a donor having a body weight of 60 kg to less than 70 kg are further selected.

### Example 4.12

### Selection of Test Cells

Among the candidate cell collections which show about 10% or lower growth rates, cell collections isolated from a donor having a body weight of 70 kg to less than 80 kg are further selected.

### Example 4.13

### Selection of Test Cells

Among the candidate cell collections which show about 10% or lower growth rates, cell collections isolated from a donor having a body weight of 80 kg to less than 90 kg are further selected.

### Example 4.14

### Selection of Test Cells

Among the candidate cell collections which show about 10% or lower growth rates, cell collections isolated from a donor having myocardial infarction are further selected.

### Example 4.15

### Selection of Test Cells

Among the candidate cell collections which show about 10% or lower growth rates, cell collections isolated from a donor having ischemic stroke are further selected.

### Example 4.16

### Selection of Test Cells

Among the candidate cell collections which show about 10% or lower growth rates, cell collections isolated from a donor having spinal cord injury are further selected.

### Example 4.17

### Selection of Test Cells

Among the candidate cell collections which show about 10% or lower growth rates, cell collections isolated from a donor having liver cirrhosis are further selected.

### Example 4.18

### Selection of Test Cells

Among the candidate cell collections which show about 10% or lower growth rates, cell collections isolated from a male donor of the age of 50 or higher are further selected.

### Example 4.19

### Selection of Test Cells

Among the candidate cell collections which show about 10% or lower growth rates, cell collections isolated from a female donor of the age of 50 or higher are further selected.

### Example 4.20

### Selection of Test Cells

Among the candidate cell collections which show about 10% or lower growth rates, cell collections isolated from a donor of the age of 50 or higher and having a body weight of 80 kg to less than 90 kg are further selected.

### Example 4.21

### Selection of Test Cells

Among the candidate cell collections which show about 10% or lower growth rates, cell collections isolated from a donor of the age of 50 or higher and having a body weight of 90 kg or higher are further selected.

### Example 4.22

### Selection of Test Cells

Among the candidate cell collections which showed about 10% or lower growth rates, cell collections isolated from a donor of the age of 50 or higher and having myocardial infarction are further selected.

### Example 4.23

### Selection of Test Cells

Among the candidate cell collections which show about 10% or lower growth rates, cell collections isolated from a donor of the age of 50 or higher and having ischemic stroke are further selected.

### Example 4.24

### Selection of Test Cells

Among the candidate cell collections which show about 10% or lower growth rates, cell collections isolated from a donor of the age of 50 or higher and having spinal cord injury are further selected.

### Example 4.25

### Selection of Test Cells

Among the candidate cell collections which show about 10% or lower growth rates, cell collections isolated from a donor of the age of 50 or higher and having liver cirrhosis are further selected.

### Example 5

### Pre-Selection of FBS

New batches of FBS were ordered from a commercial manufacturer, Gibco®. Since FBS is produced from bovine, prion that may be responsible for bovine spongiform encephalopathy (BSE) such as Creutzfeldt-Jakob disease could be present in FBS. In order to avoid a potential risk of prion, new batches of FBS should be ordered from prion-free countries such as Australia and New Zealand. The country of production was confirmed from the manufacturer's information document. Further, the manufactures generally provide an information document providing their quality control tests. Before initiating the test for FBS efficacy, the information document was reviewed to confirm that there was no bovine-associated virus or bacteria in the newly ordered batches of FBS.

### Example 6

### Selection of FBS (I)

The newly ordered batches of FBS, which were confirmed free from prion and any bovine-specific virus or bacteria, were tested using the selected test cell collections. Two collections of test cells were used to test the efficacy of new batches of FBS. The information related to the two test cell collections and the tested FBS batches is provided below:

TABLE 8
Information of the test cell collections and FBS batches on test

| Test cell | | | |
|---|---|---|---|
| collection/ | Information of test | | |
| group ID. | cells' donor | FBS batch ID. | Note |
| T34 | 54 year, male, | FBS-a (manufactured | Previously used |
| | mononuclear cells | in Australia) | FBS batch |
| | cryopreserved | | |
| T35 | 54 year, male, | FBS-b (manufactured | New batch of |
| | mononuclear cells | in Australia) | FBS |
| | cryopreserved | FBS-c (manufactured | New batch of |
| | | in Australia) | FBS |

A. Growth Rate Measurement

First, three different cell culture media, each of which comprised 10% of a different batch of FBS (i.e. FBS-a, FBS-b, and FBS-c), were prepared. The culture media were prepared using DMEM basic formulation.

Cryopreserved T34 and T35 cell collections were thawed and cultured in three different culture media for 12 days, which was referred as P0 passage. During this P0 passage, the mononuclear cells were cultured and mesenchymal stem cells (MSC) were isolated therefrom. Once this initial 12 days of culturing (P0) was done, the MSC were further cultured for five more passages during next 18 days. The cell number at each passage was counted and growth rate was graphed from the counting data. The cell counting results and growth rate data are presented below:

TABLE 9
T34 cell growth at passage P0: Isolation
of mesenchymal stem cells (MSC)

| FBS | Number of mononucleus | Number of isolated |
|---|---|---|
| batch | cells at day 0 | MSC at day 12 |
| ID. | (×10⁶) | (×10⁶) |
| FBS-a | 1.83 | 1.02 |
| FBS-b | 1.83 | 0.48 |
| FBS-c | 1.83 | 0.99 |

TABLE 10
T34 cell growth at passages P1-P5

| | Number of | Number of | Number of | Number of | Number of | Number of |
|---|---|---|---|---|---|---|
| FBS | cells at the | cells at the | cells at the | cells at the | cells at the | cells at the |
| batch | beginning of | end of P1 | end of P2 | end of P3 | end of P4 | end of P5 |
| ID. | P1 (×10⁶) | (×10⁶) | (×10⁶) | (×10⁶) | (×10⁶) | (×10⁶) |
| FBS-a | 0.48 | 2.87 | 4.59 | 16.52 | 56.16 | 162.86 |
| FBS-b | 0.48 | 1.9 | 3.04 | 5.77 | 16.73 | 46.84 |
| FBS-c | 0.48 | 2.02 | 3.43 | 9.60 | 26.88 | 80.64 |

TABLE 11
Summary of T34 cell population and growth rate

| | Number at the | Number of | Culture | | Ratio |
|---|---|---|---|---|---|
| FBS | beginning | cells at the | period | | (new batch/ |
| batch | of P1 | end of P5 | (P1 to | Growth | existing |
| ID. | (×10⁶) | (×10⁶) | P5) | rate | batch) |
| FBS-a | 0.48 | 162.86 | 18 days | 159.6 | 1 |
| FBS-b | 0.48 | 46.84 | 18 days | 97.5 | 0.6 |
| FBS-c | 0.48 | 80.64 | 18 days | 81.4 | 0.5 |

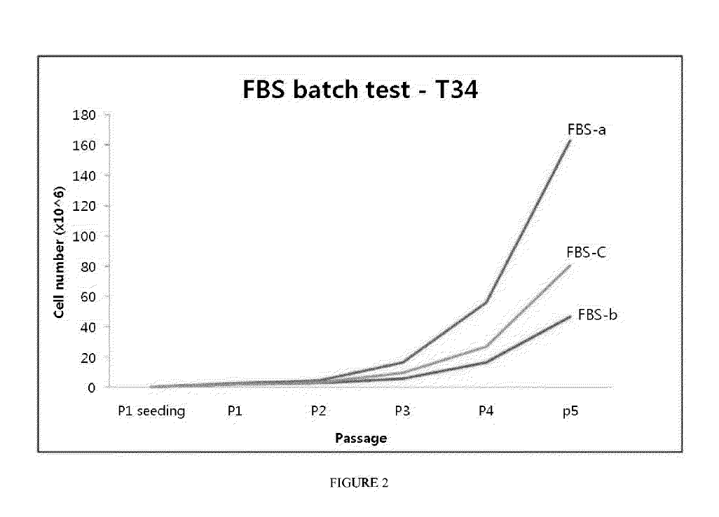

FIG. 2 shows the growth curve of T34 cells in FBS-a, FBS-b, and FBS-c in five passages (P1 to P5).

During the first 12 days of initial culturing period, T34 cell collections cultured in each of two new batches of FBS (i.e. FBS-b and FBS-c) showed about 50% of growth rate compared to T34 cells cultured in the existing FBS batch (i.e. FBS-c). Similarly, the overall growth rate after 17 days, 5 passages of culturing, the new batches of FBS still showed about only 50 to 60% of the growth rate than that of the exiting FBS-c batch.

TABLE 12
T35 cell growth at passage P0: Isolation
of mesenchymal stem cells (MSC)

| FBS | Number of mononucleus | Number of isolated |
|---|---|---|
| batch | cells at day 0 | MSC at day 12 |
| ID. | (×10⁶) | (×10⁶) |
| FBS-a | 1.83 | 0.45 |
| FBS-b | 1.83 | 0.41 |
| FBS-c | 1.83 | 0.44 |

TABLE 13
T35 cell growth at passages P1-P5

| | Number of | Number of | Number of | Number of | Number of | Number of |
|---|---|---|---|---|---|---|
| FBS | cells at the | cells at the | cells at the | cells at the | cells at the | cells at the |
| batch | beginning of | end of P1 | end of P2 | end of P3 | end of P4 | end of P5 |
| ID. | P1 (×10⁶) | (×10⁶) | (×10⁶) | (×10⁶) | (×10⁶) | (×10⁶) |
| FBS-a | 0.41 | 1.17 | 2.69 | 12.91 | 43.89 | 100.94 |
| FBS-b | 0.41 | 0.69 | 1.72 | 6.19 | 9.9 | 25.74 |
| FBS-c | 0.41 | 0.75 | 1.65 | 4.62 | 7.85 | 20.41 |

TABLE 14
Summary of T35 cell population and growth rate

| | Number at the | Number of | Culture | | Ratio |
|---|---|---|---|---|---|
| FBS | beginning | cells at the | period | | (new batch/ |
| batch | of P1 | end of P5 | (P1 to | Growth | existing |
| ID. | (×10⁶) | (×10⁶) | P5) | rate | batch) |
| FBS-a | 0.41 | 100.94 | 17 days | 224.3 | 1 |
| FBS-b | 0.41 | 25.74 | 17 days | 9.9 | 0.2 |
| FBS-c | 0.41 | 20.41 | 17 days | 7.85 | 0.2 |

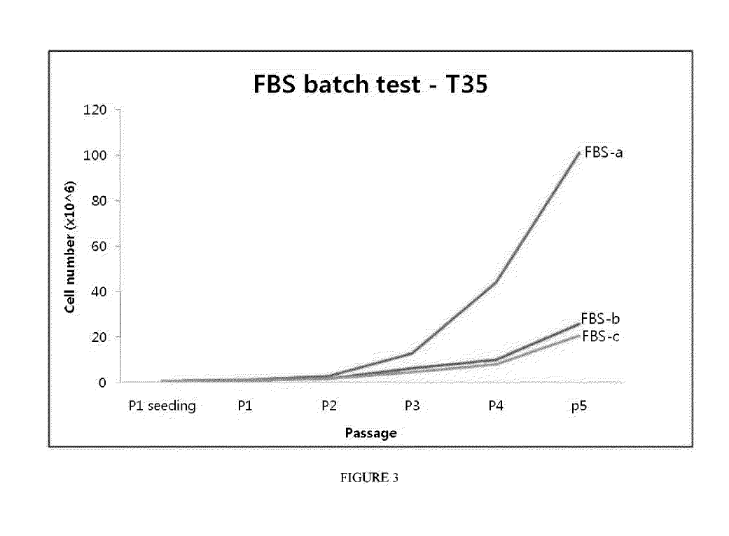

FIG. 3 shows the growth curve of T35 cells in FBS-a, FBS-b, and FBS-c in five passages (P1 to P5).

The result obtained with T35 cells was worse than that with T34 cells. While during the first 12 days of initial culturing period, the growth rate of T35 cells in all three batches of FBS (FBS-a, FBS-b, and FBS-c) was relatively similar. However, during the continuous culturing of 17 days, 5 passages, the growth rates measured from both of the new batches of FBS was significantly reduced. As a result, the cell growth rates at the end of P5 passage from the new batches of FBS-b and FBS-c were merely about 20% of that of existing FBS-a batch.

B. Cellular Morphology

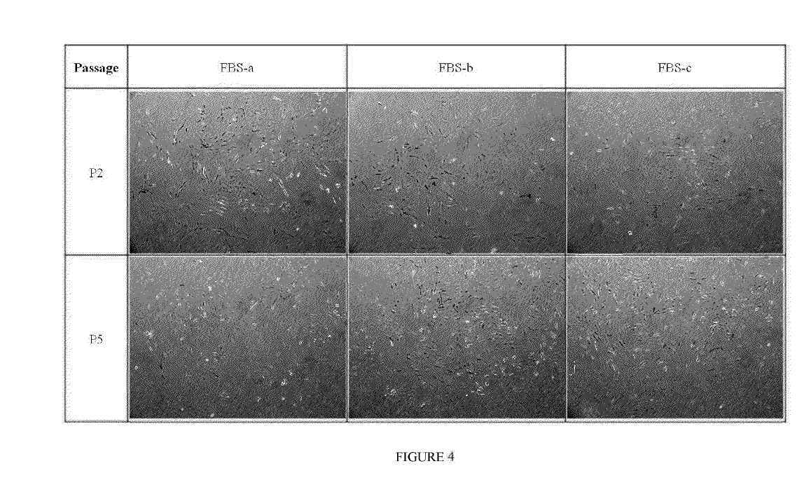

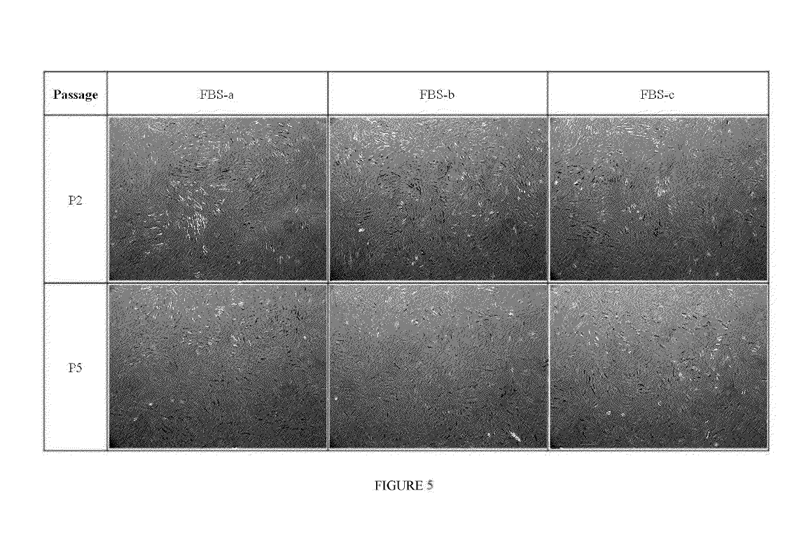

The morphology of T34 and T35 cells was monitored during the cell culture. The tested test cell collections showed a fibroblast-like morphology which is typical in mesenchymal stem cells. FIGS. 4 and 5 show representative images of cellular morphology of T34 and T35 cells.

C. Immunophenotype: Expression Profile of Cell Surface Markers

The expression profiles of various cell surface markers were monitored in T34 and T35 cells. In this example, P3 and P4 of T34 cells as well as P4 of T35 cells cultured in FBS-b and FBS-c showed less than 85% of expression of the positive marker CD73. In addition, P4 of T34 cells as well as P4 of T35 cells cultured in FBS-b showed higher than 2% expression of the negative marker CD14. However, at P5 of both T34 and T35 cells showed more than 85% expression of the positive markers such as CD73 and CD105 and less than 2% of the negative markers such as CD14, CD34, and CD45, which are generally consistent with typical characteristics of mesenchymal stem cells. The results of immunophenotype tests are provided below:

TABLE 15
Expression profile of several cell surface markers in T34 cells

| T34 | | P3 | P4 | P5 |
|---|---|---|---|---|
| FBS-a | CD105 (%) | 98.5 | 99.2 | 99.5 |
| | CD73 | 95.6 | 91.3 | 99.3 |
| | CD34 | 1.3 | 1.3 | 0.9 |
| | CD14 | 1.0 | 1.6 | 1.3 |
| | CD45 | 1.2 | 1.1 | 1.0 |
| FBS-b | CD105 (%) | 90.5 | 95.6 | 97.5 |
| | CD73 | 84.9 | 84.6 | 93.2 |
| | CD34 | 1.2 | 1.4 | 1.2 |
| | CD14 | 0.9 | 2.8 | 0.8 |
| | CD45 | 1.0 | 1.0 | 1.4 |
| FBS-c | CD105 (%) | 95.5 | 99.3 | 98.9 |
| | CD73 | 87.5 | 96.3 | 98.1 |
| | CD34 | 1.0 | 1.8 | 1.1 |
| | CD14 | 1.0 | 1.2 | 1.3 |
| | CD45 | 1.0 | 1.6 | 0.8 |

TABLE 16
Expression profile of several cell surface markers in T35 cells

| T35 | | P4 | P5 |
|---|---|---|---|
| FBS-a | CD105 (%) | 99.6 | 99.5 |
| | CD73 | 93.3 | 99.3 |
| | CD34 | 1.9 | 1.3 |
| | CD14 | 2.0 | 1.4 |
| | CD45 | 2.3 | 1.0 |
| FBS-b | CD105 (%) | 77.4 | 99.1 |
| | CD73 | 88.6 | 99.1 |
| | CD34 | 1.6 | 0.8 |
| | CD14 | 2.9 | 1.5 |
| | CD45 | 1.4 | 0.9 |
| FBS-c | CD105 (%) | 94.9 | 98.8 |
| | CD73 | 73.3 | 97.6 |
| | CD34 | 2.0 | 1.2 |
| | CD14 | 1.3 | 1.1 |
| | CD45 | 1.6 | 0.9 |

D. Conclusion

As shown above, the growth rate, cellular morphology, and immunophenotype profile of two test cell collections T34 and T35 were tested with three different batches of FBS (two new batches and one existing batch). While the cellular morphology and the immunophenotype profile of the tested cell collections were consistent with typical characteristics of mesenchymal stem cells. Further, while the data are known shown, the multipotency of T34 and T35 cells to osteoblast, adipocyte and cardiomyocyte was tested, and confirmed that all three batches of FBS shows positive results with no substantial difference among them. However, when the growth rates of T34 and T35 cells were significantly reduced in both of the new batches FBS-b and FBS-c than that of the existing FBS batch FBS-a. Accordingly, the two tested new batches of FBS were determined not suitable and efficient for cell culturing.

### Example 7

### Selection of FBS (II)

Once one or more new batches of FBS, which are free from prion or any bovine-specific virus or bacteria are pre-selected, the efficacy of pre-selected batches of FBS can be tested using the selected test cell collections.

In the following example, two test cell collections were used to test the efficacy of new batches of FBS. The information related to the two test cell collections and the tested FBS batches are provided below:

TABLE 17
Information of the test cell collections and FBS batches on test

| Test cell | | | |
|---|---|---|---|
| collection/ | Information of test | | |
| group ID. | cells' donor | FBS batch ID. | Note |
| T34 | 54 year, male, | FBS-a (manufactured | Previously used |
| | mononuclear cells | in Australia) | FBS batch |
| | cryopreserved | | |
| T43 | 60 year, female, | FBS-b (manufactured | New batch of |
| | mononuclear cells | in Australia) | FBS |
| | cryopreserved | | |

A. Growth Rate Measurement

First, two different cell culture media each of which comprised 10% of a different batch of FBS (i.e. FBS-a and FBS-b) were prepared. The culture media were prepared using DMEM basic formulation.

Cryopreserved T34 and T43 cells were thawed and cultured in three different culture media for 12 days, which is referred as P0 passage. During this P0 passage, the mononuclear cells were cultured and mesenchymal stem cells were isolated therefrom. Once this initial 12 days of culturing was done, the culture cells were further cultured for five more passages during 20 days. The cell number at each passage was counted and growth rate was graphed from the counting data. The cell counting results and growth rate data are presented below:

TABLE 18
T34 cell growth at passage P0: Isolation
of mesenchymal stem cells (MSC)

| FBS | Number of mononucleus | Number of isolated |
|---|---|---|
| batch | cells at day 0 | MSC at day 12 |
| ID. | (×10⁶) | (×10⁶) |
| FBS-a | 2.2 | 0.21 |
| FBS-b | 2.2 | 0.29 |

TABLE 19
T34 cell growth at passages P1-P5

| | Number of | Number of | Number of | Number of | Number of | Number of |
|---|---|---|---|---|---|---|
| FBS | cells at the | cells at the | cells at the | cells at the | cells at the | cells at the |
| batch | beginning of | end of P1 | end of P2 | end of P3 | end of P4 | end of P5 |
| ID. | P1 (×10⁶) | (×10⁶) | (×10⁶) | (×10⁶) | (×10⁶) | (×10⁶) |
| FBS-a | 0.21 | 1.25 | 3.37 | 11.45 | 22.9 | 52.67 |
| FBS-b | 0.21 | 0.98 | 2.64 | 8.18 | 21.26 | 44.64 |

TABLE 20
Summary of T34 cell population and growth rate

| | Number at the | Number of | Culture | | Ratio |
|---|---|---|---|---|---|
| FBS | beginning | cells at the | period | | (new batch/ |
| batch | of P1 | end of P5 | (P1 to | Growth | existing |
| ID. | (×10⁶) | (×10⁶) | P5) | rate | batch) |
| FBS-a | 0.21 | 52.67 | 20 days | 250.8 | 1 |
| FBS-b | 0.21 | 44.64 | 20 days | 212.5 | 0.85 |

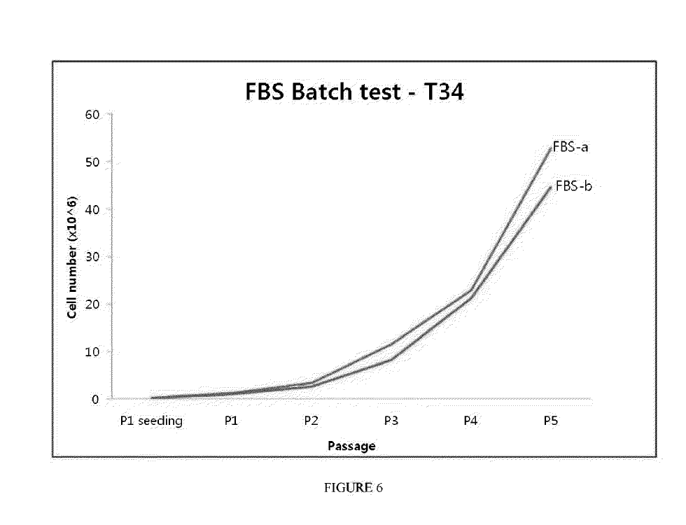

FIG. 6 shows the growth curve of T34 cells in FBS-a, FBS-b, and FBS-c in five passages (P1 to P5).

During the first 12 days of initial culturing period, T34 cells cultured in both batches of FBS-a and FBS-b showed relatively weak growth conditions. However, in the next culturing period for five passages (P1 to P5), the cell growth of T34 cells was recovered in both batches of FBS. At the final passage, the growth rate of T34 cells cultured in the new batch of FBS-b reached about 85% of that measured in the previously used batch of FBS-a.

TABLE 21
T43 cell growth at passage P0: Isolation
of mesenchymal stem cells (MSC)

| FBS | Number of mononucleus | Number of isolated |
|---|---|---|
| batch | cells at day 0 | MSC at day 12 |
| ID. | (×10⁶) | (×10⁶) |
| FBS-a | 5.7 | 0.6 |
| FBS-b | 5.7 | 1.22 |

TABLE 22
T43 Cell growth at passages P1-P5

| | Number of | Number of | Number of | Number of | Number of | Number of |
|---|---|---|---|---|---|---|
| FBS | cells at the | cells at the | cells at the | cells at the | cells at the | cells at the |
| batch | beginning of | end of P1 | end of P2 | end of P3 | end of P4 | end of P5 |
| ID. | P1 (×10⁶) | (×10⁶) | (×10⁶) | (×10⁶) | (×10⁶) | (×10⁶) |
| FBS-a | 0.6 | 2.08 | 6.15 | 15.99 | 39.97 | 115 |
| FBS-b | 0.6 | 2.52 | 7.05 | 17.62 | 66.95 | 214 |

TABLE 23
Summary of T43 Cell population and growth rate

| | Number at the | Number of | Culture | | Ratio |
|---|---|---|---|---|---|
| FBS | beginning | cells at the | period | | (new batch/ |
| batch | of P1 | end of P5 | (P1 to | Growth | existing |
| ID. | (×10⁶) | (×10⁶) | P5) | rate | batch) |
| FBS-a | 0.6 | 115 | 20 days | 191.6 | 1 |
| FBS-b | 0.6 | 214 | 20 days | 356.6 | 1.86 |

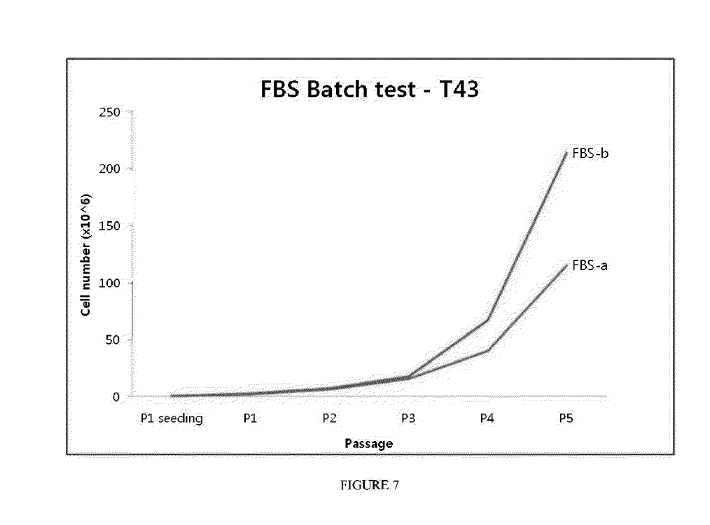

FIG. 7 shows the growth curve of T43 cells in FBS-a, FBS-b, and FBS-c in five passages (P1 to P5).

In the test using T43 cell, the cell growth rate measured in the new batch of FBS-b is higher than that measured in the existing batch of FBS-a. Therefore, based on the growth rate, the new batch of FBS appears to have improved efficacy than that of the existing FBS.

B. Cellular Morphology

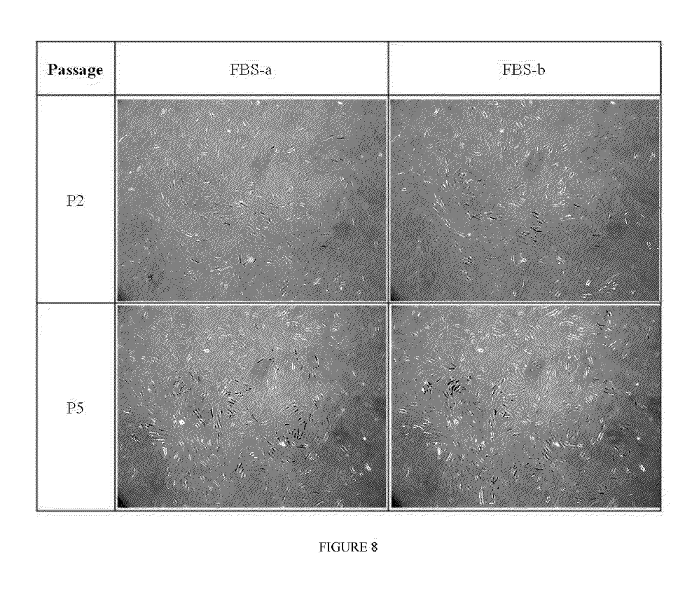

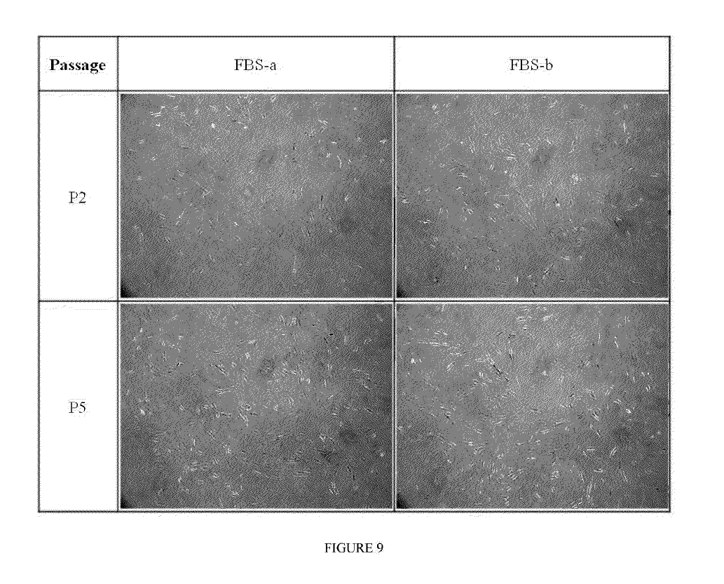

The morphology of T34 and T43 cells was monitored at every passage. The tested test cell collections showed a fibroblast-like morphology which is typical in mesenchymal stem cells. FIGS. 8 and 9 show representative images of cellular morphology of T34 and T43 cells.

C. Immunophenotype: Expression Profile of Cell Surface Markers

The expression profile of various cell surface markers was monitored in T34 and T43 cells from P2 to P5. In every tested passage of T34 and T43 cells, positive markers such as CD73 and CD105 showed more than 85% of expression profile. In addition, at every tested passage of T34 and T35 cells, the negative markers such as CD14, CD34, and CD45 showed about or less than 2% of expression profile. In both cell collections of T34 and T43, the expression of all the tested positive and negative markers was within the expression ranges of typical MSC cells. The results of immunophenotype tests are provided below:

TABLE 24
Expression profile of several cell surface markers in T34 cells

| T34 | | P2 | P3 | P4 | P5 |
|---|---|---|---|---|---|
| FBS-a | CD105 (%) | 95.4 | 97.3 | 95.0 | 99.7 |
| | CD73 | 98.1 | 99.3 | 98.7 | 100 |
| | CD34 | 0.9 | 0.8 | 1.1 | 0.7 |
| | CD14 | 1.5 | 0.8 | 1.7 | 1.3 |
| | CD45 | 1.0 | 0.9 | 1.1 | 0.2 |
| FBS-b | CD105 (%) | 97.8 | 97.1 | 97.9 | 98.8 |
| | CD73 | 98.7 | 99.4 | 99.8 | 99.8 |
| | CD34 | 1.2 | 1.1 | 0.8 | 0.9 |
| | CD14 | 2.0 | 1.2 | 1.3 | 1.5 |
| | CD45 | 1.7 | 0.8 | 0.8 | 1.0 |

TABLE 25
Expression profile of several cell surface markers in T43 cells

| T43 | | P2 | P3 | P4 | P5 |
|---|---|---|---|---|---|
| FBS-a | CD105 (%) | 99.5 | 99.4 | 99.4 | 98.6 |
| | CD73 | 97.9 | 99.5 | 99.8 | 99.1 |
| | CD34 | 1.3 | 0.7 | 1.0 | 0.8 |
| | CD14 | 1.8 | 1.2 | 1.3 | 1.4 |
| | CD45 | 1.1 | 0.9 | 0.9 | 1.0 |
| FBS-b | CD105 (%) | 99.0 | 99.4 | 99.8 | 98.8 |
| | CD73 | 98.9 | 99.5 | 99.9 | 98.8 |
| | CD34 | 1.0 | 1.5 | 1.3 | 1.0 |
| | CD14 | 1.6 | 0.8 | 0.8 | 1.0 |
| | CD45 | 0.8 | 1.4 | 0.6 | 0.9 |

D. Multipotency Test
Differentiation to Osteoblast

The differentiation potency to osteoblast was monitored at the P2 and P5 passages in T34 and T43 cells. The protocol to induce the cell fate to osteoblast is provided below:
I) 1−3×10⁵ of the cultured cells were plated into a 6-well plate and 3 mL of a fresh DMEM low glucose/10% FBS medium was provided. The cells were then cultured at 37° C., 5% CO₂ for 24 hours.
II) After 24 hours of incubation, if the cells were grown to a high density, the cell culture medium was removed. If the cells were not grown to a high density, the cells were incubated for another 24 hours and the cell culture medium was removed. After removing the culture medium, the cells were washed twice with 2 mL of DPBS and the osteoblast induction medium was provided to the cells. The composition of osteoblast induction medium is as follows:
   DMEM (Dulbecco's Modified Eagle Medium) high glucose
   10% FBS
   100 U/mL Penicillin-Streptomycin and 100 μg/mL Streptomycin
   3.7 g/L sodium bicarbonate
   0.1 μM Dexamethasone
   10 mM β-glycerol phosphate
   50 μM L-ascorbic acid
III) Once the osteoblast induction medium was provided, the cells were incubated at 37° C., 5% CO₂ for 7-14 days. During the 7-14 days of induction period, a fresh induction medium was provided every 2-3 days.
IV) After 7-14 days of induction, an alkaline phosphatase activity staining was conducted with cultured cells and the presence and/or intensity of staining were observed with or without an optical microscope. Negative and positive controls were prepared for comparison.

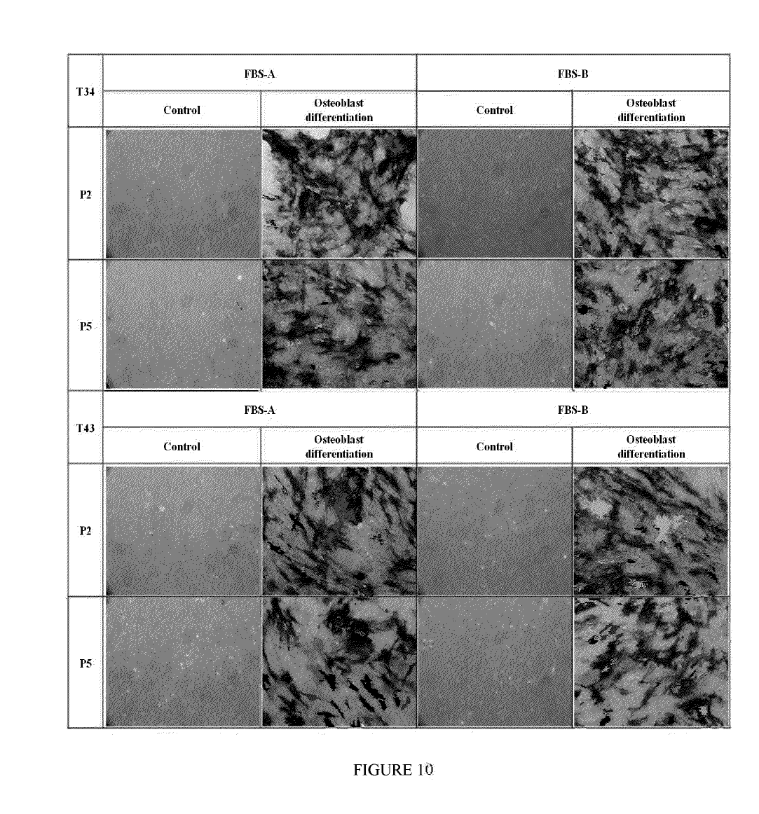

The potency of T34 and T43 cells cultured in FBS-a and FBS-b was measured as described above. Both cell collections were differentiated to osteoblast, and there was no notable difference between the different batches of FBS. The representative images showing the differentiation to osteoblast are provided in FIG. 10.
Differentiation to adipocyte

The differentiation potency to adipocyte was monitored at P1 and P5 passages in T34 and T43 cells. The protocol to induce the cell fate to adipocyte is provided below:
V) 2×10⁵ of the cultured cells were plated into a 6-well plate and 3 mL of a fresh DMEM low glucose/10% FBS medium was provided. The cells were then cultured at 37° C., 5% CO₂ for 24 hours.
VI) The cells were incubated until the cell density increased two folds. Once the cell density reached to two folds, the cells were incubated for 72 more hours.
VII) After 72 hours of incubation and possibly additional 24 hours if the cell density did not reach to a high density, the cell culture medium was removed from the cells. The cells were then washed twice with 2 mL of DPBS and 3 mL of the adipocyte induction medium was provided to the cells. The composition of the adipocyte induction medium is as follows:
   DMEM (Dulbecco's Modified Eagle Medium) high glucose
   10% FBS
   100 U/mL Penicillin-Streptomycin and 100 μg/ml Streptomycin
   3.7 g/L sodium bicarbonate
   1 μM Dexamethasone
   10 μg/mL Insulin
   100 μM Indomethacin
   0.5 mM Methyl-isobuthylxanthine
VIII) After 72 hours of incubation with the adipocyte induction medium, the adipocyte induction medium was removed from the cells. The cells were then washed twice with 2 mL of DPBS and 3 mL of the fresh adipocyte induction medium was provided to the cells. The cells were incubated for 24 hours. Steps of III) and IV) were repeated twice.
IX) After completing the repeat of steps III) and IV), the adipocyte induction medium was removed from the cells. The cells were then washed twice with 2 mL of DPBS and 3 mL of the adipocyte induction maintenance medium was provided to the cells. The composition of the adipocyte induction maintenance medium is as follows:
   DMEM (Dulbecco's Modified Eagle Medium) high glucose
   10% FBS
   100 U/mL Penicillin-Streptomycin and 100 μg/ml Streptomycin
   3.7 g/L sodium bicarbonate
   10 μg/mL Insulin
X) Once the adipocyte induction maintenance medium was provided, the cells were incubated for 24 hours. After 24 hours of incubation, the adipocyte induction maintenance medium was removed from the cells. The cells were then washed once with 2 mL of DPBS and 1 mL of neutral buffered formalin was provided to the cells to fix the cells. The incaution time was 1 hour.
XI) After the fixation, the neutral buffered formalin was removed and the cells were washed once with 2 mL of DPBS-diluted 60% 2-propanol. The cells were then stained with 2 mL of oil red O solution for 30 minutes which was followed by DPBS washing.
XII) The presence and/or intensity of staining were observed with or without an optical microscope. Negative and positive controls were prepared for comparison.

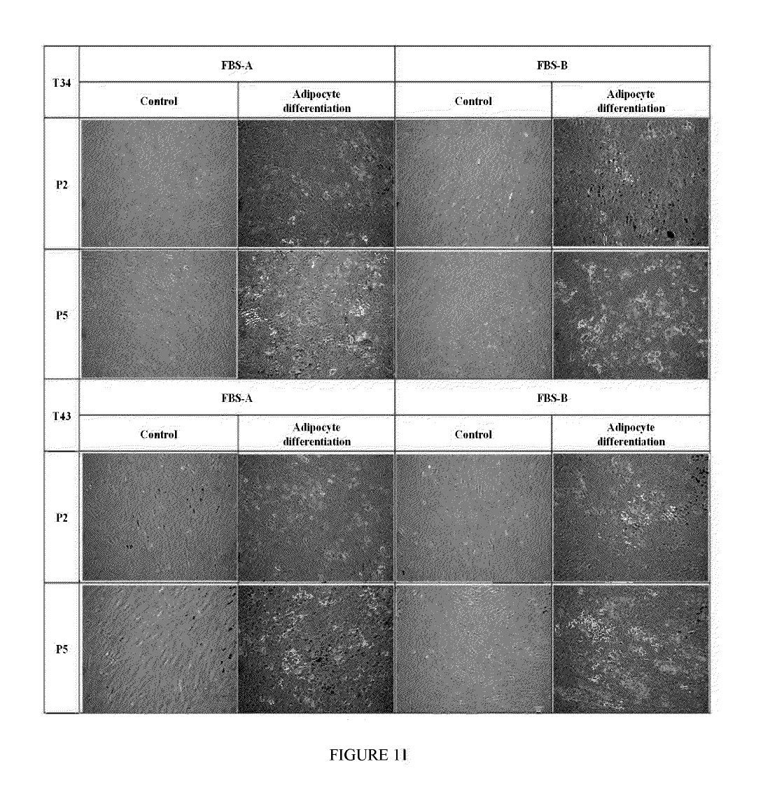

The potency of T34 and T43 cells cultured in FBS-a and FBS-b was measured as described above. Both cell collections were differentiated to adipocyte, and there was no notable difference between the different batches of FBS. The representative images showing the differentiation to adipocyte are provided in FIG. 11.
Differentiation to cardiomyocyte

The differentiation potency to cardiomyocyte was monitored at the P5 passages in T34 and T43 cells. The protocol to induce the cell fate to cardiomyocyte is provided below:
XIII) 1−3×10⁵ of the cultured cells were plated into a 6-well plate and 3 mL of a fresh DMEM low glucose/10% FBS medium was provided. The cells were then cultured at 37° C., 5% CO₂ for 24 hours.
XIV) After 24 hours of incubation, the cells were plated into chamber slide and cultured 2-3 days. After 2-3 days of incubation, culture medium was removed. After removing the culture medium, the cells were washed twice with 1 mL of DPBS and the cardiomyocyte induction medium was provided to the cells. The composition of cardiomyocyte induction medium is as follows:
   DMEM (Dulbecco's Modified Eagle Medium) low glucose
   10% FBS
   100 U/mL Penicillin-Streptomycin and 100 μg/mL Streptomycin
   3.7 g/L sodium bicarbonate
   10 μM 5-azacytidine
   10 μg/mL bFGF
XV) Once the cardiomyocyte induction medium was provided, the cells were incubated at 37° C., 5% CO₂ for 48 hours.
XVI) After 48 hours of incubation with the cardiomyocyte induction medium, the cardiomyocyte induction medium was removed from the cells. The cells were then washed twice with 1 mL of DPBS and 1 mL of the fresh cardiomyocyte induction medium lacking 5-azacytidine was provided to the cells. The cells were incubated for 7 to 14 days. During the 7-14 days of incubation, the fresh cardiomyocyte induction medium lacking 5-azacytidine was provided every 3-4 days.
XVII) The cells were washed with PBS and fixed with 10% formalin solution for immunostaining.
XVIII) After the fixation, the formalin solution was removed from the cells and the cells were washed with PBS. The washed cells were then added to a PBS solution containing 0.1% Triton X-100 and incubated for an hour. This step is for permeabilizaiton.
XIX) After the permeabilizaiton step, the cells were washed and a PBS solution containing 5% BSA was added thereto for blocking. The blocking step was conducted for an hour.
XX) After the blocking step, the cells were washed with PBS, and a solution containing troponin T antibody (1:500 dilution) was added thereto. The cells were then incubated at 4° C. overnight.
XXI) After the overnight incubation, the cells were washed with PBS, and a solution containing a secondary antibody (1:2000 dilution) was added thereto. The cells were then incubated at room temperature for an hour.
XXII) After an hour of incubation with the secondary antibody solution, the cells were washed with PBS. A mounting solution was added to the cells and a cover slip was applied to cover the cells. The presence and/or intensity of immunostaining from the prepared cells were observed under a fluorescent microscope.

The potency of T34 and T43 cells cultured in FBS-a and FBS-b was measured as described above. Both cell collections were differentiated to cardiomyocyte, and there was no notable difference between the different batches of FBS. The representative images showing the differentiation to cardiomyocyte are provided in FIG. 7.

E. Conclusion

The two test cell collections of T34 and T43 were used to evaluate the new batch of FBS-b. As shown above, the growth rate and biological characteristics of T34 and T43 cells were not substantially different between the media containing FBS-a or FBS-b. Rather, FBS-b showed a higher growth rate when T43 cells was tested as compared to the existing batch of FBS-a. Further, the cellular morphology and immunophenotype were consistent with typical characteristics of mesenchymal stem cells. As for the multipotency of the culture cells, both cells were shown to maintain the multipotency during the culture process. Accordingly, the new batch of FBS-b was proven to have a similar or even better efficacy in growing test cell collections. From this evaluation test, the new batch of FBS-b was identified as being suitable and efficient for culturing mesenchymal stem cells and could be used for primary stem cell culture.

1. A method of selecting a collection of cells for choosing a fetal bovine serum (FBS) to use in stem cell transplantation, the method comprising:
providing a plurality of collections of cells, cells of each collection being of a different source from cells of another of the plurality of collections;
monitoring growth of cells in the plurality of collections;
providing information associated with each collection, the information is selected from the group consisting of:
the age of a subject from which the collection of cells was obtained,
the weight of a subject from which the collection of cells was obtained, the
gender of a subject from which the collection of cells was obtained,
the type of tissues of the subject from which the collection of cells was obtained,
information about the current and previous health condition of the subject from which the collection of cells was obtained,
family medical history of the subject from which the collection of cells was obtained, and
the number of culturing passages of the collection after the collection of cells was originally obtained from the subject,
selecting a first one among the plurality of collections based on either or both of the monitoring of cell growth and further based on the information associated with each collection, wherein the first collection is selected as one of a bottom 50% group of collections, wherein the bottom 50% group consists of collections showing 50% or less in cell growth among the plurality of collections;
providing at least part of the first collection of cells that has been selected for test-growing in a plurality of fetal bovine sera, from which to choose one.

2. The method of claim 1, wherein the selection of the first collection is based on that the first collection of cells is one of a least-growing group of collections, wherein the collections of the least-growing group are bottom 10% in cell growth among the plurality of collections.

3. The method of claim 1, wherein the selection of the first collection is based on that the first collection of cells is the least-growing collection in cell growth compared to the other collections.

4. The method of claim 1, wherein the selection of the first collection is based on that the first collection of cells was obtained from the subject at the age of 80 or older.

5. The method of claim 1, wherein the selection of the first collection is based on that the first collection of cells was obtained from bone marrow of the subject.

6. The method of claim 1, wherein the selection of the first collection is based on that the first collection of cells was obtained from the subject when the subject was medically diagnosed of a disease.

7. The method of claim 1, wherein the selection of the first collection is based on that the first collection of cells was obtained from the subject having history of a disease selected from the group consisting of myocardial infarction, ischemic stroke, spinal cord injury, and liver cirrhosis.

8. The method of claim 1, wherein the selection of the first collection is based on that the first collection of cells is:
one of a least-growing group of collections, wherein the collections of the least-growing group are bottom 10% in cell growth among the plurality of collections; and
obtained from the subject at the age of 80 or older.

9. The method of claim 1, wherein the selection of the first collection is based on that the first collection of cells is:
one of a least-growing group of collections, wherein the collections of the least-growing group are bottom 10% in cell growth among the plurality of collections; and
obtained from the subject when the subject was medically diagnosed of a disease.

10. The method of claim 1, wherein the selection of the first collection is based on that the first collection of cells is:
one of a least-growing group of collections, wherein the collections of the least-growing group are bottom 10% in cell growth among the plurality of collections; and
obtained from the subject having history of a disease selected from the group consisting of myocardial infarction, ischemic stroke, spinal cord injury, and liver cirrhosis.

11. The method of claim 1, wherein the selection of the first collection is based on that the first collection of cells is:
one of a least-growing group of collections, wherein the collections of the least-growing group are bottom 10% in cell growth among the plurality of collections;
obtained from the subject at the age of 80 or older; and
obtained from the subject when the subject was medically diagnosed of a disease.

12. A method of selecting a fetal bovine serum (FBS) for use in culturing stem cells for transplantation, the method comprising:
providing a collection of cells selected by the method of claim 1;
providing a plurality of FBS samples;
formulating a plurality of culture media, each comprising one of the plurality of FBS samples;
culturing the cells of the selected collection in the plurality of culture media;
monitoring growth of the cells of the selected collection in the plurality of culture media;
based on monitoring, identifying one or more culture media, among the plurality of culture media, that has shown at least 1.5 fold cell growth after one culturing passage; and
selecting at least one FBS that is included in the one or more identified culture media for use in culturing stem cells for transplantation.

13. The method of claim 12, further comprising:
prior to selecting at least one FBS, identifying at least one culture medium, among the selected one or more culture media, that has shown at least 142 fold cell growth after five culturing passages.

14. The method of claim 12, further comprising:
prior to selecting at least one FBS, identifying at least one culture medium, among the selected one or more culture media, that has shown at least 100 fold cell growth after five culturing passages.

15. The method of claim 12, further comprising:
prior to selecting at least one FBS, identifying at least one culture medium, among the selected one or more culture media, in which the cells of the selected collection have shown a plastic-adherent, fibroblast-like morphology.

16. The method of claim 12, further comprising:
prior to selecting at least one FBS, identifying at least one culture medium, among the selected one or more culture media, in which the cells of the selected collection have shown a high level of expression of CD105, and CD73 and a low level of expression of CD45, CD34, CD14.

17. The method of claim 12, further comprising:
prior to selecting at least one FBS, identifying at least one culture medium, among the selected one or more culture media, in which the cells of the selected collection have potency to differentiate into at least one selected from the group consisting of osteoblast, adipocyte and cardiomyocyte.

18. The method of claim 17, wherein said identifying comprises further culturing the cells of the selected collection to determine if the cells differentiate into at least one of osteoblast, adipocyte and cardiomyocyte.

19. A culture medium for culturing stem cells for transplantation, the culture medium comprising;
an FBS selected by the method of claim 12; and
a basal medium.

20. A method of stem cell transplantation, the method comprising:
providing mononuclear cells for culturing;
providing a culture media comprising an FBS selected by the method of claim 12;
culturing the mononuclear cells in the culture media to produce mesenchymal stem cells; and
transplanting the produced mesenchymal stem cells to a patient.

21. The method of claim 20, wherein providing the mononuclear cells comprises obtaining the mononuclear cells from the same patient.
